(19) United States
(12) Patent Application Publication
(10) Pub. No.: US 2026/0216211 A1
(43) Pub. Date: Jul. 30, 2026

(54) COMPOSITION COMPRISING ESTETROL AND DROSPIRENONE FOR USE IN RELIEVING AND TREATING PAIN

(71) Applicant: EATETRA SRL, Liège (BE)

(72) Inventors: Masashi HIRAYAMA, Liège (BE); Masayoshi NOGAMI, Liège (BE)

(73) Assignee: EATETRA SRL, Liège (BE)

(21) Appl. No.: 19/159,121

(22) PCT Filed: Feb. 16, 2024
(86) PCT No.: PCT/EP2024/053977

§ 371 Date: Aug. 22, 2025

(30) Foreign Application Priority Data
Feb. 23, 2023 (EP) 23158334.5
Sep. 1, 2023 (EP) 23194864.7

(51) Int. Cl. A61K 31/585; A61K 31/565; A61P 15/00

(52) U.S. Cl. CPC A61K 31/585; A61K 31/565; A61P 15/00

(57) ABSTRACT

The present invention relates to a pharmaceutical composition comprising estetrol and drospirenone for use in relieving pain associated with endometriosis in a subject. The pharmaceutical composition described herein is particularly effective in relieving pain associated with endometriosis in subjects that report a high degree, frequency, and/or intensity of pain, such as measured by the Visual Analogue Scale (VAS) score, and/or have a further complication such as adenomyosis or uterine fibroids. Also described herein are uses related to the above and corresponding methods of treatment.

### FIELD OF THE INVENTION

The present invention broadly relates to the field of medicine, and more particularly to hormone treatments of female subjects that are characterized by endometriosis associated pain. Specifically, the invention relates to a composition comprising estetrol and drospirenone for use in relieving (i.e. reducing or treating) pain, especially pelvic pain in a safe and efficient manner in female subjects. The present invention is particularly effective for treating endometriosis subjects that have a high Visual Analog Scale (VAS) score.

### BACKGROUND OF THE INVENTION

Endometriosis is a medical condition characterized by the uncontrolled growth of endometrial tissue engrafts outside of the uterine cavity in female subjects, causing pain and sometimes even infertility or (ovarian) cancer. The exact cause is still under investigation, although recently evidence is increasing that endometriosis is caused by multiple factors. Associations have been described with impaired immunity, localized hormonal influences, genetics, and even environmental contaminants (Zondervan et al., N Engl J Med, 2020). Although several screening tools and tests have been proposed and tested, none are currently validated to accurately identify or predict individuals or populations that are most likely to have the disease. Therefore, histological and/or laparoscopic confirmation remains necessary to arrive at a conclusive diagnosis. Early suspicion of endometriosis is a key factor for early diagnosis, as endometriosis can often present symptoms that mimic other conditions and contribute to a diagnostic delay.

Due to the difficult diagnosis for healthcare practitioners, defining an exact incidence remains challenging. However, certain studies suggest that up to 11% of women of reproductive age in the general population may be affected by the condition (Shafrir et al., Best Pract Res Clin Obstet Gynaecol, 2018). In 2021, the WHO stated that endometriosis has significant social, public health and economic implications decreasing quality of life due to severe pain and other symptoms. At present, there is no curative treatment available for the condition. Management of symptoms is therefore of crucial importance to improve the quality of life of subjects affected by the condition.

Certain symptomatic treatment strategies to manage endometriosis-induced pain have been described in the art and include the administration of non-steroidal anti-inflammatory medicaments, analgesics, and surgery to remove endometriotic tissue, adhesions, and scar tissue. However, each of these strategies are characterised by highly variable success rates, a considerable risk of (severe) side-effects, long-term safety concerns, and combinations thereof (Johnson et al., Hum Reprod, 2013).

Recently, the use of YAZ Flex (comprising 20 μg ethinylestradiol and 3 mg DRSP intended for use in a flexible extended-cycle oral contraceptive regimen) for endometriosis-associated pelvic pain was studied (clinicaltrials.gov, study identifier NCT03126747) and proven to be effective to manage endometriosis-associated pain.

Nevertheless, there remains an unmet need for improved innovative strategies for the management of the disease, especially for those that provide a safe and effective reduction of endometriosis-induced pain to further improve the quality of life of a subject afflicted by endometriosis and having less side-effects than the existing products.

### SUMMARY OF THE INVENTION

As evidenced by the examples which illustrate certain representative embodiments of the invention, the inventors have found that a composition comprising from 13.5 mg to 16.5 mg estetrol (E4) and from 2.5 mg to 3.5 mg drospirenone (DRSP) is particularly suited for relieving a subject from endometriosis-associated pain. Unexpectedly, this composition was observed to be superior to existing hormonal endometriosis therapies for relieving pain, such as a composition comprising ethinylestradiol (EE) and drospirenone (DRSP), especially in subjects that report, or are considered to have, a high degree of pain (i.e. a high Visual Analogue Scale score) caused by said endometriosis. This improvement could also be observed in subjects suffering from adenomyosis and/or uterine fibroids in addition to endometriosis. Hence, in a more general context the composition specified herein is particularly suited to alleviate endometriosis and endometriosis symptoms.

More specifically, a similar decrease of the VAS for the most severe endometriosis-associated pelvic pain was observed between the treatment group (E4/DRSP) and the control group (YAZ Flex; EE/DRSP). Surprisingly, E4/DRSP was shown to be significantly more effective than the control group in suppressing pain during the non-withdrawal bleeding period particularly in patients with a pre-administration baseline VAS value of 70 mm or more. In combination with the general excellent estetrol safety profile that has been described at numerous occasions throughout the art when compared to other estrogens, it may therefore be concluded that endometriosis subjects with a baseline VAS of 70 mm or greater should ideally use the tested E4/DRSP composition instead of other hormonal compositions that are used to date in order to manage endometriosis pain during the non-withdrawal bleeding period.

The invention therefore provides the following aspects:
Aspect 1. A pharmaceutical composition comprising from about 13.5 mg to about 16.5 mg estetrol or hydrates of estetrol and from about 2.5 mg to about 3.5 mg drospirenone, for use in relieving pain associated with endometriosis in a subject and/or for use in treating endometriosis in said subject.
Aspect 2. A pharmaceutical composition comprising from about 13.5 mg to about 16.5 mg estetrol or hydrates of estetrol and from about 2.5 mg to about 3.5 mg drospirenone for use in treating a subject suffering from pelvic pain and/or for use in treating endometriosis in said subject.
Aspect 3. A pharmaceutical composition comprising from about 13.5 mg to about 16.5 mg estetrol or hydrates of estetrol and from about 2.5 mg to about 3.5 mg drospirenone, for use in relieving pain associated with endometriosis in a subject and/or for use in treating endometriosis in said subject, wherein the use comprises a step of determining whether the patient has pelvic pain prior to administration defined by a Visual Analogue Scale (VAS) score of greater than about 40 mm, about 50 mm, about 60 mm, or about 70 mm.
Aspect 4. Use of a composition comprising from about 13.5 mg to about 16.5 mg estetrol or hydrates of estetrol and from about 2.5 mg to about 3.5 mg drospirenone, for the manufacture of a medicament for relieving pain associated with endometriosis in a subject or for the manufacture of a medicament for treating endometriosis.
Aspect 5. Use of a composition comprising from about 13.5 mg to about 16.5 mg estetrol or hydrates of estetrol and from about 2.5 mg to about 3.5 mg drospirenone for the manufacture of a medicament for treating a subject suffering from pelvic pain.
Aspect 6. Use of a composition comprising from about 13.5 mg to about 16.5 mg estetrol or hydrates of estetrol and from about 2.5 mg to about 3.5 mg drospirenone for the manufacture of a medicament for relieving pain associated with endometriosis in a subject or for the manufacture of a medicament for treating endometriosis in said subject, wherein the use comprises the step of determining whether the patient has pelvic pain prior to administration as defined by a VAS score of greater than about 40 mm, about 50 mm, about 60 mm, or about 70 mm.
Aspect 7. A method of relieving pain associated with endometriosis in a subject or for treating endometriosis in said subject, comprising administration of a composition comprising from about 13.5 mg to about 16.5 mg estetrol or hydrates of estetrol and from about 2.5 mg to about 3.5 mg drospirenone to said subject.
Aspect 8. A method of treating a subject suffering from pelvic pain, comprising administration of a composition comprising from about 13.5 mg to about 16.5 mg estetrol or hydrates of estetrol and from about 2.5 mg to about 3.5 mg drospirenone to said subject.
Aspect 9. A method of relieving pain associated with endometriosis in a subject or of treating endometriosis in said subject, comprising a first step of determining whether the patient has pelvic pain defined by a VAS score of greater than about 40 mm, about 50 mm, about 60 mm, or about 70 mm, and comprising a further step of administrating a composition comprising from about 13.5 mg to about 16.5 mg estetrol and from about 2.5 mg to about 3.5 mg drospirenone to said subject.
Aspect 10. The pharmaceutical composition for use according to any one of aspects 1 to 3, the use according to any one of aspects 4 to 6, or the method according to any one of aspects 7 to 9, wherein the estetrol is estetrol monohydrate.
Aspect 11. The pharmaceutical composition for use, the use, or the method according to any one of the preceding aspects, wherein the pain in the subject is relieved (i.e. reduced) during the non-withdrawal bleeding period.
Aspect 12. The pharmaceutical composition for use, the use, or the method according to any one of the preceding aspects, wherein the pain in the subject is relieved during the period not related to the withdrawal bleeding period, preferably during a period up to two days before the onset of the withdrawal bleeding.
Aspect 13. The pharmaceutical composition for use, the use, or the method according to any one of the preceding aspects, wherein the pain in the subject is relieved during a period of from two days before the onset of the withdrawal bleeding to the onset of the withdrawal bleeding, preferably during a period of from one day before the onset of the withdrawal bleeding to the onset of the withdrawal bleeding.
Aspect 14. The pharmaceutical composition for use, the use, or the method according to any one of the preceding aspects, wherein the pain in the subject is relieved from about 48 hours prior to onset of the withdrawal bleeding until the onset of the withdrawal bleeding, preferably wherein the pain in the subject is relieved from about 36 hours, more preferably from about 24 hours prior to onset of the withdrawal bleeding until the onset of the withdrawal bleeding.
Aspect 15. The pharmaceutical composition for use, the use, or the method according to any one of the preceding aspects, wherein the pain in the subject is relieved on hormone-free days that mark the end of an administration cycle of a hormonal contraceptive, preferably wherein the pain in the subject is relieved on days prior to day 25 (first day of the hormone-free interval) in a typical hormonal contraceptive treatment schedule.
Aspect 16. The pharmaceutical composition for use, the use, or the method according to any one of the preceding aspects, wherein the subject is not considered to have or not diagnosed to have primary dysmenorrhea, or wherein the anamnesis of the subject resulted in exclusion of primary dysmenorrhea in said subject, or wherein the subject is a subject wherein primary dysmenorrhea is excluded by the anamnesis of said subject.
Aspect 17. The pharmaceutical composition for use, the use, or the method according to any one of the preceding aspects, wherein the subject has pelvic pain as defined by a VAS score of greater than about 40 mm, about 50 mm, about 60 mm, or about 70 mm prior to administration.
Aspect 18. The pharmaceutical composition for use, the use, or the method according to any one of the preceding aspects, wherein the subject is suffering from pelvic pain associated with endometriosis having a VAS score of greater than about 40 mm, about 50 mm, about 60 mm, or about 70 mm prior to administration.
Aspect 19. The pharmaceutical composition for use, the use, or the method according to any one of the preceding aspects, wherein the subject is suffering from pelvic pain induced by endometriosis having a VAS score of greater than about 40 mm, about 50 mm, about 60 mm, or about 70 mm prior to administration.
Aspect 20. The pharmaceutical composition for use, the use, or the method according to any one of the preceding aspects, wherein the pain is resulting from endometrial tissue engrafts outside the uterine cavity in the pelvis.
Aspect 21. The pharmaceutical composition for use, the use, or the method according to aspect 20, wherein the pain is resulting from endometrial tissue engrafts located on the ovaries, fallopian tubes, tissues that hold the uterus in place (ligaments), outer surface of the uterus, vagina, cervix, vulva, bowel, bladder, rectum, or any combination thereof.
Aspect 22. The pharmaceutical composition for use, the use, or the method according to aspect 20 or 21, wherein said pain is resulting from endometrial tissue engrafts located on the ovaries, fallopian tubes, tissues that hold the uterus in place (ligaments), outer surface of the uterus, or any combination thereof.
Aspect 23. The pharmaceutical composition for use, the use, or the method according to any one of the preceding aspects, wherein said pain is chronic pelvic pain, pelvic pain such as lower abdominal pain and/or low back pain, defecation pain and sexual intercourse pain, or pain caused by adhesion of endometrium in the Douglas pouch.
Aspect 24. The pharmaceutical composition for use, the use, or the method according to any one of the preceding aspects, wherein the use results in an improvement of the CGI-I Scale (Clinician Global Impressions-Improvement Scale) of at least about 10%, preferably at least about 20%, more preferably of about 28.9%.
Aspect 25. The pharmaceutical composition for use, the use, or the method according to any one of the preceding aspects, wherein the use results in an improvement graded “significantly satisfied or more” of the PGI-I Scale (Patient Global Impressions-Improvement Scale) of at least about 10%, preferably at least about 20%, more preferably of about 24%.
Aspect 26. The pharmaceutical composition for use, the use, or the method according to any one of the preceding aspects, wherein the use results in a responder proportion of at least about 40%, preferably at least about 50%, more preferably of at least about 60%.
Aspect 27. The pharmaceutical composition for use, the use, or the method according to any one of the preceding aspects, wherein the use results in a decrease of CA125, preferably wherein the use results in a decrease of CA125 serum levels to below 35 U/ml.
Aspect 28. The pharmaceutical composition for use, the use, or the method according to any one of the preceding aspects, wherein the subject is diagnosed as having endometriosis by laparotomy/laparoscopy, and/or as having ovarian chocolate cysts as assessed by Transvaginal Ultrasound (TVUS) and/or Magnetic Resonance Imaging (MRI).
Aspect 29. The pharmaceutical composition for use, the use, or the method according to any one of the preceding aspects, wherein the subject has adenomyosis and/or uterine fibroids in addition to endometriosis.
Aspect 30. The pharmaceutical composition for use, the use, or the method according to aspect 29, wherein the subject has adenomyosis which affects about 25% or less of the uterine corpus (mild adenomyosis), or from about 25% to about 50% of the uterine corpus (moderate adenomyosis), or more than about 50% of the uterine corpus (severe adenomyosis).
Aspect 31. The pharmaceutical composition for use, the use, or the method according to aspect 29, wherein the subject has uterine fibroid(s) classified as pedunculated, submucosal, intramural, subserosal, or any combination thereof.
Aspect 32. The pharmaceutical composition for use, the use, or the method according to any one of the preceding aspects, wherein the composition is used in cycles of 21 to 28 daily active dosage units of said composition.
Aspect 33. The pharmaceutical composition for use, the use, or the method according to aspect 32, wherein the composition is used in cycles of 24 daily active dosage units of said composition.
Aspect 34. The pharmaceutical composition for use, the use, or the method according to aspect 32 or 33, wherein said cycle comprises an administration-free interval of 7 days, preferably an administration-free interval of 4 days.
Aspect 35. The pharmaceutical composition for use, the use, or the method according to any one of aspects 32 to 34, wherein use of the composition results in a decrease in VAS score from prior to administration to after the second or third administration cycle.
Aspect 36. The pharmaceutical composition for use, the use, or the method according to aspect 35, wherein use of the composition results in a decrease in VAS score of at least about 10%, preferably at least about 20%, more preferably at least about 30%, more preferably at least about 40%, most preferably at least about 50% from prior to administration to after the second or third administration cycle.
Aspect 37. The pharmaceutical composition for use, the use, or the method according to aspect 36, wherein said decrease is maintained substantially stable after the second or third administration cycle.
Aspect 38. The pharmaceutical composition for use, the use, or the method according to any one of the preceding aspects, wherein use of the composition results in improvement in the severity of Douglas induration, restriction of uterine mobility and/or pelvic tenderness, reduced size and/or number of ovarian chocolate cysts as assessed by TVUS or MRI.
Aspect 39. The pharmaceutical composition for use, the use, or the method according to aspect 38, wherein the use of the composition results in an improvement in the severity of at least about 10%, preferably at least about 20%, more preferably at least about 30%, more preferably at least about 40%, most preferably at least about 50%.
Aspect 40. The pharmaceutical composition for use, the use, or the method according to any one of the preceding aspects, wherein use of the composition results in less TEAE than use of an EE 20 μg/DRSP 3 mg, fixed dose combination tablet.
Aspect 41. The pharmaceutical composition for use, the use, or the method according to any one of the preceding aspects, wherein said subject has reduced Treatment Emergent Adverse Events (TEAE) selected from the group consisting of: intermenstrual bleeding, headache, nausea and heavy menstrual bleeding.
Aspect 42. The pharmaceutical composition for use, the use, or the method according to any one of the preceding aspects, for reducing endometriosis-associated sleep disturbance.

In any one of the aspects defined herein, said dosage unit may be presented as a kit-of-parts containing a packaging unit, e.g. a blister pack, containing the daily oral dosage units comprising the estetrol and drospirenone. The skilled person will additionally know that, within the scope of the present invention, each packaging unit, e.g. blister pack, may be numbered or otherwise marked. In a particular embodiment of the kit-of-parts, the packaging unit comprises 28 containers or a multitude of 28 containers, such as 2 to 12 times 28 containers. In a preferred embodiment, the packaging unit comprises 3 or 6 times 28 containers. Within the scope of the invention, each packaging unit may be a sealed blister pack with a cardboard, paperboard, foil plastic backing and enclosed in a suitable cover.

Also envisaged in any one of the aspects defined herein are packaging units such as bottles. The material of the bottle is not particularly limiting. In preferred embodiments, the bottle is a glass bottle characterized by a color capable of reducing or preventing degradation of the contents of the bottle by e.g. UV light while maintaining a degree of transparency that allows for visual inspection of the contents of said bottle. Suitable colors include without limitation amber, cobalt, or vintage green.

The above and further aspects and preferred embodiments of the invention are described in the following sections and in the appended claims. The subject matter of the appended claims is hereby specifically incorporated in this specification.

### BRIEF DESCRIPTION OF THE FIGURES

FIG. 1. (A) Individual VAS values and Spline curves. X-axis: relative day from the first treatment day; Y-axis: VAS value (mm). (B) Mean VAS values. X-axis: days; Y-axis: VAS value (mm). “FSN-013” corresponds to the 15 mg E4/3 mg DRSP group.

FIG. 2. Definition of Responders. Responder: VAS reduced by more than 70 mm in 80% or more days of evaluated period. Evaluated period: non-withdrawal bleeding period between days 29-84.

FIG. 3. CGI-I and PGI-I improvement in the responder and non-responder (E4/DRSP+Yaz Flex). Left panel: CGI-I; right panel: PGI-I. Diagonally striped: Responder, right sample group of each histogram. Solid grey filled: Non-Responder, left sample of each histogram.

FIG. 4. Proportion of the responder in E4/DRSP and Yaz Flex groups. FSN-013 group corresponds to 15 mg E4/3 mg DRSP group.

FIG. 5. (A) Gynecologic objective findings following 6 cycle treatment—proportion of subjects who were improved, stable or worsened: induration of cul-de sac (p=0.002, Fisher's exact), limitation of uterine mobility (p=0.005, Fisher's exact), pelvic tenderness (p=0.022, Fisher's exact). (B) Stratified analysis with or without adenomyosis in alteration of gynecologic objective findings following 6 cycle treatments.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g. any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e. also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Unless indicated otherwise, all methods, steps, techniques and manipulations that are not specifically described in detail can be performed and have been performed in a manner known per se, as will be clear to the skilled person. Reference is for example again made to standard handbooks as well as to the general background art referred to herein and to the further references cited therein.

The term “estetrol” as used herein refers to 1,3,5 (10)-estratrien-3,15alpha,16alpha,17beta-tetrol or 15alpha-hydroxyestriol as well as hydrates of estetrol, e.g. estetrol monohydrate. “Estetrol”, or short “E4” is an estrogen steroid produced by the foetal human liver (PubChem CID: 27125). Estetrol may be described as a 3-hydroxy steroid corresponding to 17beta-estradiol wherein the 15a and 16a positions are substituted for two additional hydroxy groups. It is known that estetrol is an estrogen receptor agonist (Coelingh Bennink et al., Climacteric, 2008). The estetrol may be chemically synthetised, synthesised by the use of (mutant) recombinant enzymes, or synthesised by any combination thereof. It is therefore evident that the terms “estetrol” and “estetrol components” equally encompass further chemically modified estetrol. Estetrol may be indicated in the art by its molecular formula: C₁₈H₂₄O₄, or by structural formula (I).

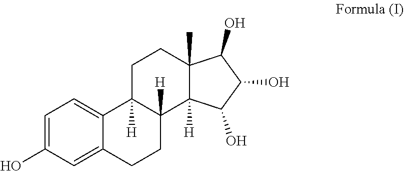

It is understood that when estetrol is mentioned throughout any section of this specification, any estetrol-containing component (i.e. compound) and/or estetrol derivative (such as an estetrol ester) is also envisaged. More preferably, in the context of the present disclosure, a particularly preferred estetrol (component) is estetrol monohydrate. A skilled person appreciates that estetrol monohydrate corresponds to estetrol containing one molecule of water, and that the core structural formula of estetrol does not differ from Formula (I). By means of illustration and not limitation, the structural formula of estetrol monohydrate is indicated by Formula (II):

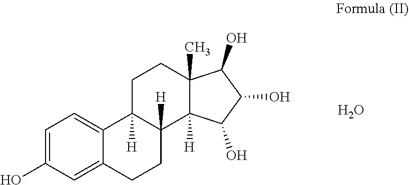

The compositions, uses, and methods described throughout the present disclosure are generally compared in terms of safety and efficacy to a fixed dose combination tablet comprising ethinylestradiol and drospirenone. “Ethinylestradiol” (abbreviated as EE, PubChem CID: 5991; molecular formula: C₂₀H₂₄O₂), refers to an estrogen distinct from estetrol and estetrol monohydrate that is widely used in birth control pills in combination with a progestogenic component. Ethinylestradiol is a synthetic derivative of estradiol (the latter being a natural estrogen). The popularity of ethinylestradiol is partly due to its favourable properties when compared to estradiol including improved bioavailability and an increased resistance to metabolism. By means of illustration and not limitation, the structural formula of ethinylestradiol is indicated by Formula (III):

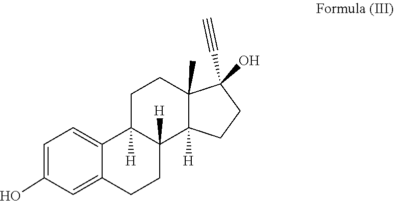

“Drospirenone” (abbreviated as DRSP, PubChem CID: 68873) is an example of a progestogenic component and enjoys a widespread use in Combined Oral Contraceptives (commonly abbreviated as COCs) due to its antimineralocorticoid and antiandrogenic activity combined with a general low off-target activity. In general, drospirenone-containing COCs are referred to as fourth generation COCs. Non-limiting examples of commercially available COCs comprising drospirenone are known as “YaZ™” and Yasmin™. An illustrative example of a drospirenone only progestogen pill is “Slynd™”, which is also commercially available. Additionally, hormone replacement therapy compositions comprising an estrogen such as estradiol and drospirenone are available such as “Angeliq™”. Drospirenone may alternatively be indicated in the art by its molecular formula C₂₄H₃₀O₃, or by the structural formula (IV):

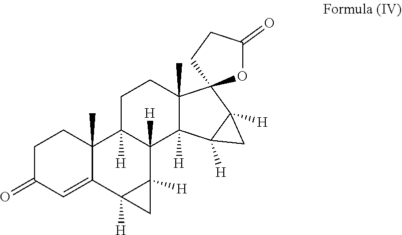

It is understood that when the term “drospirenone” is used herein, any drospirenone derivatives are also envisaged. The terms “progestogen”, “gestagen”, or “gestogen” and derived hereof “progestogenic components” as used both herein and in the art refer to any molecule that produces effects similar to those of the natural female sex hormone progesterone in the body of a subject. Progestogens are considered to be agonists of the progesterone receptors and their functions have been thoroughly examined in the art (inter alia discussed in Kuhl, Climacteric, 2005). Progestins are a subgroup of progestogens that comprise synthetic progestogens. While the above terms may be used interchangeably in the art, there is a general understanding that when progestin is mentioned, synthetic progestogens are meant.

Unexpectedly, the inventors have found that administration of a composition comprising estetrol and drospirenone represents a highly effective yet safe manner to treat endometriosis-induced and/or endometriosis-associated pain in a subject. Moreover, said combination provides distinct advantages when compared to other hormonal treatment strategies that are generally recommended for this indication, such as but not limited to compositions comprising ethinylestradiol and drospirenone, which may be administered in a flexible extended-cycle oral contraceptive regimen. The improvements are particularly observed in subjects that are characterised by high degrees of (i.e. intense and/or frequent) pain induced by endometriosis. This is unexpected, since a skilled person would assume that estrogens combined with drospirenone would generally lead to similar treatment results. Especially given the fact that estetrol is generally considered a “weak” estrogen (Gérard et al., J Endocrinol, 2015), a more potent effect when compared to ethinylestradiol is remarkable. The general safety reported throughout the art for combined oral contraceptives with estetrol as the estrogen component could be confirmed by the minimal effect of the estetrol/drospirenone combination on the blood coagulation and fibrinolysis system, and limited Treatment-Emergent Adverse Events (TEAEs). No deviations of significance from baseline could be observed for a large number of clinical markers such as haematological parameters, biochemical tests, urine tests, electrocardiogram (ECG), and QT interval.

Hence, in a first aspect the invention relates to a pharmaceutical composition comprising estetrol and drospirenone for use in relieving pain associated, caused, and/or induced by endometriosis. The estetrol and drospirenone are typically comprised in the pharmaceutical composition at pharmaceutically effective amounts, such as from about 13.5 mg to about 16.5 mg of estetrol and from about 2.5 mg to about 3.5 mg of drospirenone. Alternatively worded, the invention envisages the use of a composition comprising about 13.5 mg to about 16.5 mg estetrol and about 2.5 mg to about 3.5 mg drospirenone for the manufacture of a medicament for relieving pain associated with endometriosis in a subject. Yet alternatively worded, the invention envisages a method of relieving pain associated with endometriosis in a subject, wherein the method comprises a step of administering to a subject a composition comprising about 13.5 mg to about 16.5 mg estetrol and about 2.5 mg to about 3.5 mg drospirenone. Use of the pharmaceutical composition as described herein is envisaged for relieving pain associated with endometriosis. Optionally, use of the pharmaceutical composition as described herein is envisaged for relieving pain induced by endometriosis.

“Endometriosis” as used herein relates to a benign (i.e. non-malignant) proliferative disorder in which functioning endometrial tissue is present in a location in the body other than the endometrium of the uterus, i.e. outside the uterine cavity. As a skilled person thus appreciates, endometriosis is a distinct medical condition from endometrial cancer(s). Endometrial cancer is not encompassed by the term “endometriosis” used herein. Endometriosis may develop when cells lining the uterus are implanted at distal sites which can include the pelvic area, peritoneal surfaces, ovaries, ligaments, bowel, and bladder. Hence, the term “endometriosis” encompasses any form or specific classification of the conditions including but not limited to: endometriosis externa, endometrioma, endometriotic nodules other than of the uterosacral ligaments, autoimmune endometriosis, mild endometriosis, moderate endometriosis, severe endometriosis, superficial (peritoneal) endometriosis, deep (invasive) endometriosis, and ovarian endometriosis. Unless explicitly specified, the term endometriosis is therefore used herein to describe any of these conditions. It is further evident to a skilled person that the efficacy of the composition subject of the present disclosure for treating pain associated with endometriosis also essentially implies that the composition is effective for treating endometriosis as such. Optionally, alleviation of the pain associated with endometriosis as described herein may therefore equally indicate treatment of endometriosis as such.

“Adenomyosis” is when tissue similar to the lining of the uterus (endometrium) starts to grow into the muscle wall of the uterus (myometrium), i.e. endometrial-like tissue growing into the uterus muscle. It causes the uterus to thicken and enlarge—sometimes, up to double or triple its usual size. Adenomyosis can cause painful periods, heavy or prolonged menstrual bleeding with clotting and abdominal/pelvic pain. Adenomyosis may cause painful menstrual cramps (dysmenorrhea), heavy menstrual bleeding (menorrhagia), abnormal menstruation, pelvic pain with or without severe cramping, painful intercourse (dyspareunia), infertility, enlarged uterus, bloating or fullness in the belly (adenomyosis belly). Adenomyosis may be diagnosed by pelvic exam, transvaginal ultrasound and imaging scans such as magnetic resonance imaging (MRI) scans. Adenomyosis is generally classified into mild, moderate, or severe adenomyosis based on the portion of affected uterine corpus. By means of illustration and not limitation, mild adenomyosis corresponds to a portion of affected uterine corpus of about 25% or less, moderate adenomyosis corresponds to a portion of affected uterine corpus from about 25% to about 50%, and severe adenomyosis corresponds to a portion of affected uterine corpus of more than about 50%.

“Uterine fibroids” are noncancerous growths of the uterus that often appear during childbearing years. Also called leiomyomas or myomas, uterine fibroids are not associated with an increased risk of uterine cancer and almost never develop into cancer. Fibroids range in size from seedlings, undetectable by the human eye, to bulky masses that can distort and enlarge the uterus. It is possible to have a single fibroid or multiple ones. In extreme cases, multiple fibroids can expand the uterus so much that it reaches the rib cage and can add weight.

Fibroids can be diagnosed during a pelvic exam or prenatal ultrasound. The most common symptoms of uterine fibroids are heavy menstrual bleeding, menstrual periods lasting more than a week, pelvic pressure or pain, frequent urination, difficulty emptying the bladder, constipation and backache or leg pains. Fibroids are generally classified by their location. Intramural fibroids grow within the muscular uterine wall. Submucosal fibroids bulge into the uterine cavity. Subserosal fibroids project to the outside of the uterus. In addition, pedunculated uterine fibroids have been described that are characterized by an attachment to the uterine wall by a stalk-like structure. More specific classification methods for uterine fibroids are available to a skilled person by their publication in the art such as the FIGO (International Federation of Gynecology and Obstetrics) classification system (Munro et al., Int J Gynaecol Obstet, 2018). Each of these different uterine fibroid types are envisaged in the context of the present invention. A skilled person further appreciates that endometriosis is a medical condition which is clearly distinguishable from dysmenorrhea, and particularly from primary dysmenorrhea. It is important to note that there are no physical findings associated with primary dysmenorrhea and that primary dysmenorrhea is not associated with any laboratory abnormalities or abnormal findings on imaging studies. Hence, a medical practitioner can readily distinguish endometriosis from primary dysmenorrhea due to the presence of endometrial tissue outside of the uterine cavity of a subject in endometriosis, which is absent in primary dysmenorrhea (e.g. Harada, Dysmenorrhea and endometriosis in young women, Yonago Acta Med, 2013).

Endometriosis may be classified according to severity, extent, location, or any combination thereof. Different stages of endometriosis may be specified (i.e., stage I-IV), and their location is important for determining the appropriate treatment regimen which may include surgical removal of the endometriotic tissues and hormonal therapies which include progestins, oral contraceptives, and GnRH antagonists. Hence, as envisaged by the present disclosure, the individual may have been identified as having stage 1 or stage 2 endometriosis. Stage 1 (or minimal) endometriosis is present a stage of the disease wherein the subject is characterised by a low number of (relatively small) implants, small wounds, and/or lesions. Said implants may be found on or in the organs or the tissue lining the pelvis or abdomen, with little to no scar tissue. Stage 2 (or mild) endometriosis is generally characterized by more implants when compared to stage 1, which can be located deeper in the tissue, and, optionally, scar tissue can be present. Alternatively, the individual may have been identified as having stage 3 or 4 endometriosis. Stage 3 (or moderate) endometriosis is generally characterized by numerous deep implants, optionally including small cysts on one or both ovaries, and thick bands of scar tissue (i.e. adhesions). Stage 4 (or severe) endometriosis is generally characterized by numerous deep implants and thick adhesions, and additionally large cysts on one or both ovaries. Thus, the subject may optionally be characterised by stage 1, stage 2, stage 3, or stage 4 endometriosis, or any combination thereof.

Alternatively, the endometriosis may be grouped according to any other classification method or classification system reported in the art. By means of illustration and not limitation, suitable systems include the rASRM classification system (American Society for Reproductive Medicine, Fertil Steril, 1997), the endometriosis fertility index (EFI) (Adamson and Pasta, Fertil Steril, 2010), and the ENZIAN score (Haas et al., Fertil Steril, 2011).

Pain is a submodality of somatic sensation resulting from a complex constellation of unpleasant sensory, emotional and cognitive experiences provoked by real or perceived tissue damage and manifested by certain autonomic, psychological, and behavioural reactions (Terman and Bonica, Bonica's management of pain, 2003). The term “pain” as used within the context of the present disclosure refers to physical pain. However, it is to be understood that physical pain may additionally cause secondary unwanted emotional states that are associated with non-physical, i.e. mental pain. Hence, while the inventors envisage a wide applicability of the herein presented compositions and methods, they may additionally exert an improvement in the emotional state of the subject, as described in detail further herein. Particularly relevant in this context is the alleviation of chronic pain, which is known to be harmful and often detrimental for the mental well-being of a subject (Sheng et al., Neural Plasticity, 2017).

The expression “to relieve” may be interchangeably used with any synonym as known in the art. Non-limiting examples of synonyms include “to alleviate”, “to treat”, “to mitigate”, “to soothe”, “to soften”, “to palliate”, “to ease”, “to reduce”, “to lessen”, and “to diminish”. Each of these terms are to be interpreted as both the therapeutic treatment of pain associated with endometriosis that has already developed, leading to (clinical) manifestations, as well as prophylactic or preventive measures, wherein the goal of the treatment is to prevent, lessen, or reduce the chances of incidence of an undesired affliction, such as to prevent occurrence, development and progression of pain associated with endometriosis. Beneficial or desired clinical results may include without limitation diminishment of the extent (i.e. a reduction in severity) of the pain associated with or induced by endometriosis, stabilized (i.e. not worsening) of said pain, delay or slowing of pain associated with endometriosis, and the like. “Prevention” or “prevent” as used in the context of the invention refers to an aversion of manifestation of a condition or disease image in a subject, i.e. the establishment of preventive measures or prophylactic measures. Preventive treatment refers to treatments wherein the object is to avoid a subject's body or an element thereof to show (worsening of) symptoms of an undesired physiological or psychological change, in the context of the present disclosure pain associated with endometriosis. As used herein, the terms “therapeutic treatment” or “therapy” and the like, refer to treatments wherein the aim is to change the perception of pain associated with endometriosis to a more desired state, such as a less severe state (e.g., amelioration, or even back to its normal, healthy state (i.e. no pain sensation), to keep it (i.e. not worsening of the pain) at said undesired physiological status (e.g. stabilization), or slow down progression to a more severe or worse perception of pain. In a specific embodiment, measurable lessening includes any statistically significant decline in a measurable marker or symptom. Statistically significant as used herein refers to p values below 0.05, which is a commonly accepted cut-off score in statistical analysis as a skilled person appreciates.

The terms “pharmaceutical formulation”, “pharmaceutical composition”, or “pharmaceutical preparation” may be used interchangeably herein. Likewise, the terms “formulation”, “composition”, or “preparation” may be used interchangeably herein. Throughout this description, absolute quantities as referred to herein correspond to the amounts present in one administration dose, unless explicitly stated otherwise. Optionally, the estetrol and drospirenone are the sole (i.e. single, only) pharmaceutically active ingredients part of the composition. The term “pharmaceutically active ingredient”, interchangeably used throughout the present disclosure with “pharmaceutically active agent” is to be interpreted according to the definition of the term by the World Health organisation: “a substance used in a finished pharmaceutical product (FPP), intended to display pharmacological activity or to otherwise have direct effect in the diagnosis, cure, mitigation, treatment or prevention of disease, or to have direct effect in restoring, correcting or modifying physiological functions in human beings”.

The term “subject”, or “patient” as used herein refers to female human subjects, preferably female subjects of reproductive age. Alternatively, peri- and/or post-menopausal female subjects are also envisaged. The female subject envisaged herein may be a subject in need of, or deemed in need of a treatment for relieving endometriosis-associated pain, or predicted to be in need of such a treatment in a foreseeable future point in time optionally due to entering a particular stage of life, such as for example the reproductive age, or in view of endometriosis in close family members, such as the mother, grandmother, or siblings. Optionally, the subject is a female subject of from about 12 to about 95 years old, preferably of from about 14 to about 80 years old, more preferably of from about 16 to about 70 years old, most preferably of from about 18 to about 60 years old. Optionally, the female subject is a subject of about 60 years or younger, preferably a subject of about 55 years or younger, preferably a subject of about 50 years or younger, preferably a subject of about 45 years or younger, preferably a subject of about 40 years or younger, preferably a subject of about 35 years or younger, preferably a subject of about 30 years or younger, preferably a subject of about 25 years or younger, preferably a subject of about 20 years or younger. Most preferably, the female subject is a subject of from about 16 to about 25 years old.

A further aspect of the invention relates to a pharmaceutical composition comprising about 13.5 mg to about 16.5 mg estetrol and from about 2.5 mg to about 3.5 mg drospirenone for use in treating a subject suffering from pelvic pain. “Pelvic pain” as used herein refers to pain in the pelvic area.

Alternatively worded, the invention relates to use of a composition comprising about 13.5 mg to about 16.5 mg estetrol and about 2.5 mg to about 3.5 mg drospirenone for the manufacture of a medicament for treating a subject suffering from pelvic pain. Yet alternatively worded, the invention relates to a method of treating a subject suffering from pelvic pain, comprising administration of a composition comprising about 13.5 mg to about 16.5 mg estetrol and about 2.5 mg to about 3.5 mg drospirenone to said subject. Optionally, the pelvic pain is characterized by a severity that limits normal (i.e. healthy) functioning of the subject in society (e.g. school and/or work absence). Optionally, the pelvic pain is diagnosed to be, or considered to be chronic pelvic syndrome (i.e. pelvic pain lasting over six months and which is of such a severity to limit functioning of the subject).

In yet a further aspect, the invention relates to a pharmaceutical composition comprising about 13.5 mg to about 16.5 mg estetrol and from about 2.5 mg to about 3.5 mg drospirenone for use in relieving pain associated with endometriosis in a subject, wherein the use comprises a step of determining whether the patient has pelvic pain prior to administration defined by a VAS score of greater than about 40 mm. Alternatively worded, the invention relates to the use of a composition comprising about 13.5 mg to about 16.5 mg estetrol and about 2.5 mg to about 3.5 mg drospirenone for the manufacture of a medicament for relieving pain associated with endometriosis in a subject, wherein the use comprises the step of determining whether the patient has pelvic pain prior to administration as defined by a VAS score of greater than about 40 mm. Preferably, the pelvic pain is defined by a VAS score of greater than 50 mm. More preferably, the pelvic pain is defined by a VAS score of greater than about 70 mm, yet more preferably greater than about 80 mm, most preferably greater than about 90 mm. Alternatively, the pelvic pain may be defined by a VAS score of from 40 mm to 100 mm, 50 mm to 80 mm, 60 mm to 80 mm, 40 mm to 70 mm, or 60 mm to 90 mm.

The term “Visual Analog Scale score”, abbreviated as “VAS score” refers to a broadly accepted pain rating scale (originally described by Hayes and Patterson in 1921). In atypical VAS, the pain score is determined by measuring the distance (mm) on the 10-cm line between the “no pain” anchor and the patient's mark, providing a range of scores from 0-100. A higher score indicates greater pain intensity. Generally, the following groups of pain intensity have been formed: no pain (0-4 mm), mild pain (5-44 mm), moderate pain (45-74 mm), and severe pain (75-100 mm). Different VAS structures, configurations, orientations, and response options have been described and are within the knowledge of the skilled person (Reviewed for example in Byrom et al., Ther Innov Regul Sci, 2022). The particular VAS scoring system that is used is therefore not particularly limiting for the invention. By means of illustration and not limitation, the VAS can be presented as a numerical rating scale with a centre point, graduations, and/or numbers; curvilinear analog scales, box-scales consisting of circles positioned at equal distance from each other, and graphic rating scales. All orientations of the scale on form sheets or electronic displays are envisaged. Alternative pain rating scales are also envisaged by the present disclosure. By means of illustration and not limitation these include the numeric rating scale (NRS-11), the Stanford pain scale, visual rating scale (VRS), or the visual numeric scale. Broader concepts of pain measurement instruments and methodologies have been described in detail in the art (e.g. in Younger et al., Current Pain And Headache Reports, 2010), each with values on the respective scale representing equivalent levels of pain in the subject. Optionally, the subject may be a subject characterized by an NRS-11 score of from 1 to 10, 2 to 9, 3 to 7, or 4 to 6. Alternatively, the subject may be a subject characterized by a Stanford pain scale score of from 1 to 3 (i.e. mild pain), from 4 to 6 (i.e. moderate pain), or from 7 to 10 (i.e. severe pain). In the context of the present disclosure, the endometriosis associated pain is preferably expressed by means of the VAS scale, as has been evaluated and recommended in the art (e.g. Bourdel et al., Hum Reprod Update, 2015). A person skilled in the art is capable of comparing reported pain degrees between different pain scales. By means of illustration and not limitation, it is generally accepted within the technical field that in a context of endometriosis an NRS value of 0 corresponds to a VRS indication of “no pain”, an NRS value of 1 to 3 corresponds to a VRS indication of “mild pain”, an NRS value of 4 to 6 corresponds to a VRS indication of “severe pain”, and an NRS value of 7 to 10 corresponds to a VRS indication of “severe pain”. Similarly, a VAS score of about 0 mm corresponds to a VRS indication of “no pain”, and a VAS score of about 100 mm corresponds to a VRS indication of “worst pain imaginable” (Bourdel et al., Hum Reprod Update, 2015).

The pain in the subject may be measured using the VAS pain score during the non-withdrawal bleeding period. In such embodiments, the pain in the subject is measured using the VAS pain score in the time window characterized by absence of withdrawal bleeding (i.e. scheduled bleeding/spotting episode). Optionally, the pain in the subject is measured using the VAS pain score at a time point at least one week, at least two weeks, or at least three weeks after cessation of the withdrawal bleeding. Optionally, the subject has pain as defined by a VAS score of greater than about 40 mm, about 50 mm, about 60 mm, about 70 mm, about 80 mm, or about 90 mm prior to administration. Alternatively, the subject has pelvic pain as defined by a VAS score of greater than about 40 mm, about 50 mm, about 60 mm, about 70 mm, about 80 mm, or about 90 mm prior to administration. Yet alternatively, the subject may be suffering from pelvic pain associated with endometriosis having a VAS score of greater than about 40 mm, about 50 mm, about 60 mm, about 70 mm, about 80 mm, or about 90 mm prior to administration.

“Withdrawal bleeding” as used herein is the vaginal bleeding that occurs during the hormone free interval part of a typical dosing schedule for hormonal contraceptives. The compositions according to the present invention are particularly suited for relieving pain in a subject during the non-withdrawal bleeding period. In some embodiments, said pain is not associated with the withdrawal bleeding period, or in other words, pain is concerned that occurs before day 25 (wherein day 25 corresponds to the first day of the hormone free interval in a typical hormonal contraceptive treatment schedule). Withdrawal bleeding usually starts around day 26. In preferred embodiments, pain is concerned that occurs on any of the days 1, 2, 3, 4, 5, 6, 7, 8, 9, 10, 11, 12, 13, 14, 15, 16, 17, 18, 19, 20, 21, 22, 23, 24.

With withdrawal bleeding period or scheduled bleeding, bleeding/spotting from day 25 (first day of hormone free interval) up to day 4 of the subsequent cycle, including any bleeding/spotting starting prior to Day 25 and continuing into the scheduled bleeding period, or bleeding/spotting that started in the scheduled bleeding period but continued after day 4 is meant. Non-withdrawal bleeding period, vice versa, refers to the days of the cycle that are different from the withdrawal bleeding period or scheduled bleeding. Spotting referred to as minimal vaginal blood loss that did not require the new use of sanitary protection, including pantyliners and/or bleeding referred to as vaginal blood loss that required the use of sanitary protection with a tampon, pad or pantyliner can also occur during the non-withdrawal bleeding period. Even episodes of bleeding/spotting can occur during the non-withdrawal bleeding period, by which one or more consecutive bleeding/spotting days bounded on either end by two bleeding/spotting free days are meant. Unscheduled bleeding/spotting refers to any bleeding/spotting that does not meet the criteria for scheduled bleeding. Even if bleeding and/or spotting occurs outside of the withdrawal or scheduled bleeding period, the period outside the scheduled bleeding period is still referred to here as the non-withdrawal bleeding period, even if the bleeding and/or spotting occurs in an episode.

### Definitions Used for Analysis of Bleeding Patterns:

| Term | Definition |
|---|---|
| Spotting | Minimal vaginal blood loss that did not require the new use |
| | of sanitary protection, including pantyliners |
| Bleeding | Vaginal blood loss that required the use of sanitary |
| | protection with a tampon, pad or pantyliner |
| Bleeding/ | One or more consecutive bleeding/spotting days bounded |
| spotting | on either end by two bleeding/spotting free days |
| episode | |
| Scheduled | Bleeding/spotting from day 25 (first day of hormone free |
| bleeding | interval) up to day 4 of the subsequent cycle, including any |
| | bleeding/spotting starting prior to Day 25 and continuing |
| | into the scheduled bleeding period, or bleeding/spotting |
| | that started in the scheduled bleeding period but continued |
| | after Day 4 |
| Unscheduled | Any bleeding/spotting not meeting criteria for scheduled |
| bleeding/ | bleeding |
| spotting | |

Thus, the subject may be a subject that has been diagnosed with endometriosis, considered to have endometriosis, or predicted to develop endometriosis (associated pain). Optionally, the subject may be diagnosed with, considered to have, or predicted to develop adenomyosis and/or uterine fibroids. The subject may be prognosticated to develop pain associated with, and/or induced by endometriosis. “Diagnosed with”, “diagnosing”, and “diagnosis” are indicative for a process of recognizing, deciding on, or concluding on a disease, condition, or (adverse side effect) in a subject on the basis of symptoms and signs and/or from results of various diagnostic procedures. “Diagnosis of” endometriosis and/or pain associated by endometriosis may particularly mean that the subject has a high, or even definite probability of experiencing respectively endometriosis or pain associated by endometriosis by a skilled medical practitioner. A subject may be diagnosed as not having such despite displaying one or more conventional symptoms or signs reminiscent of such. “Prognosticating” in the context of the invention is indicative for anticipation on the progression of pain associated with and/or induced by endometriosis in a subject and the prospect (e.g. the probability, duration, and/or extent) of recovery, and/or the severity of experiencing or amelioration of said pain associated with and/or induced by endometriosis in a subject. The term may encompass anticipation of not further worsening or aggravating of such, preferably within a given time period.

Optionally, the subject is a subject that is diagnosed as having endometriosis, and optionally adenomyosis and/or uterine fibroids, by laparotomy or laparoscopy. A skilled person is familiar with the terms “laparotomy” and “laparoscopy”, which respectively refer to a procedure wherein a surgical incision of considerable size is made into the abdominal cavity and a procedure wherein a small incision is made (also indicated in the art as a “keyhole incision” and “minimally invasive surgery”). The subject may be a subject that is diagnosed as having ovarian chocolate cysts. It is generally appreciated that the term “ovarian chocolate cysts”, interchangeably indicated throughout the art by the term “ovarian endometriomas”, refers to ovarian sacs or pouches filled with the fluid(s) present in the lining of the uterus (i.e. the endometrium). The ovarian chocolate cysts may have been detected by Transvaginal Ultrasound (TVUS) and/or Magnetic Resonance Imaging (MRI), as described in detail in the art (e.g. Bausic et al., Diagnostics (Basel), 2022).

Related to the foregoing, “predicting” or “prediction” generally refers to a statement, declaration, indication or forecasting of a disease or condition in a subject not (yet) showing any, or a limited, clinical manifestation of pain associated with and/or induced by endometriosis. A prediction of the development of said pain in a subject may indicate a probability, chance, or risk that said subject will develop said clinical manifestation, for example within a certain time period after diagnosis of the one or more endometriosis-associated symptoms. Said probability, chance or risk may be indicated as any suitable qualitative or quantitative expression, wherein non-limiting examples of a quantitative expression include absolute values, ranges or statistics. Alternatively, probabilities, chances, or risks may be indicated relative to a suitable control subject or group of control subject (i.e. a control subject population (such as, e.g., relative to a general, normal or healthy subject or subject population)). Therefore, any probability, chance or risk may be advantageously indicated as increased or decreased, upregulated or downregulated, as fold-increased or fold-decreased relative to a suitable control subject or subject population, or relative to a baseline value which may be derived from either a control subject (population) or textbook reference values. It is evident that when a population of subjects is used to define the baseline value, said baseline value will be a center size of one or more values (parameters) of a population, such as the mean or median of said value. A skilled person further appreciates that monitoring may be applied in the course of a medical treatment of a subject, such as those described by the present disclosure. Such monitoring may be comprised, e.g., in decision making whether a patient may be discharged from a controlled clinical or health practice environment, needs a change in treatment or therapy, or requires hospitalization.

The pharmaceutical composition subject of the disclosure comprises from about 13.5 mg to about 16.5 mg of estetrol and from about 2.5 mg to about 3.5 mg of drospirenone. It is to be appreciated that these ranges represent pharmaceutically effective amounts of both estetrol and drospirenone. A skilled person is aware that terms such as “quantity”, “amount” and “level” are synonyms and have a well-defined meaning in the art. Optionally, the pharmaceutical composition subject of the disclosure comprises from about 14 mg to about 16 mg of estetrol and from about 2 mg to about 3 mg of drospirenone. Optionally, the pharmaceutical composition subject of the disclosure comprises from about 14.5 mg to about 15.5 mg of estetrol and from about 2 mg to about 3 mg of drospirenone. Optionally, the pharmaceutical composition subject of the disclosure comprises about 15 mg of estetrol and about 3 mg of drospirenone. Preferably, the pharmaceutical composition subject of the disclosure comprises about 15 mg of estetrol monohydrate and about 3 mg of drospirenone.

Each of the compositions described herein may alternatively be described in relationship to the daily amounts of estetrol (monohydrate) and drospirenone that are eventually administered to a subject. Thus, any composition expressed to comprise from about 13.5 mg to about 16.5 mg of estetrol and from about 2.5 mg to about 3.5 mg of drospirenone, may equally be a composition that is administered in such an amount to correspond to a daily amount equivalent to from about 13.5 mg to about 16.5 mg of estetrol and from about 2.5 mg to about 3.5 mg of drospirenone. Hence, the expression “ . . . administered at a daily amount equivalent to . . . ” indicates the administration of a substance, in the context of the present invention estetrol and drospirenone, that effectuates the same physiological and/or psychological effects as a situation wherein the subject would have been administered said amount of estetrol and drospirenone. Additionally, the term “daily” indicates that the recited amounts are the cumulative amount that is administered to a subject per day. A skilled person understands that if the composition subject of the present disclosure is administered only once per day (i.e. daily), that the amount of estrogen administered in that single administration will be the daily dose. Alternatively, a skilled person appreciates that if the composition is administered more than once per day (e.g. 2 times or 3 times) the daily amount will correspond to the (independent) sum of all estetrol and all drospirenone administered during each administration event within a total time window of 24 hours. It is within the capacities of a skilled person to verify the daily amounts of estetrol and drospirenone.

The pharmaceutical composition described herein may be formulated into one or more dosage units. Optionally, the dosage unit is a daily dosage unit. Optionally, the dosage unit is an oral dosage unit. In a further embodiment, the dosage unit is a daily oral dosage unit. It is evident that any of the compositions and dosage units may suitably contain one or more pharmaceutically acceptable excipients. The term “pharmaceutically acceptable” as used herein is consistent with the art and means compatible with the other ingredients of a pharmaceutical composition and not deleterious to the recipient thereof.

The composition subject of the present invention is particularly suited for formulation as an oral dosage unit. However, equally envisaged are dosage units formulated towards alternative administration methods such as but not limited to sublingual, buccal, or sublabial dosage units.

“Dosage unit”, interchangeably used with “dosage form” herein indicates a physical preparate that is suitable for administration to a subject, without the necessity to adapt the preparate prior to administration, i.e. the final beneficial product. A dosage unit therefore indicates a ready-to-administer composition. The term is not limiting for any other particulars of the treatment, such as frequency of administration and/or any characteristic of the dosage unit (taste, appearance, size, etc.). In the context of the present invention, each dosage unit preferably comprises estetrol and drospirenone in an amount equivalent to from about 13.5 mg to about 16.5 mg of estetrol and from about 2.5 mg to about 3.5 mg drospirenone as pharmaceutically acceptable ingredients. The presence of estetrol and drospirenone as pharmaceutically acceptable ingredient does not exclude the presence of one or more further pharmaceutically active ingredients. By means of illustration and not limitation, a further pharmaceutically acceptable ingredient may be an analgesic, i.e. an ingredient that is able to achieve analgesia in a subject. The term “analgesic” may be used interchangeably with synonyms such as “painkiller” or “pain reliever”. Optionally the analgesic may be selected from the group consisting of: acetaminophen (i.e. paracetamol), nonsteroidal-inflammatory drugs (NSAIDs), opioids, muscle-relaxants, anti-anxiety agents, antidepressants, anticonvulsants, and corticosteroids.

The term “pharmaceutically acceptable” as used herein is consistent with the art and means compatible with the other ingredients of a pharmaceutical composition and not deleterious to the recipient thereof. Non-limiting suitable excipients are described further throughout the disclosure. Related hereto, the term “oral dosage unit” encompasses any dosage unit that is intended to and/or suitable for administration to a subject by means of the oral cavity. (Immediate or near-immediate) ingestion of the dosage unit is envisaged as an embodiment of the invention but is by no means limiting for the scope of the invention.

The oral dosage unit described herein may be a solid or semi solid dosage unit such as a tablet, a capsule, a cachet, a pellet, a pill, powder, or granules, or any combination thereof. For example, the oral dosage unit subject of the invention may be a tablet comprising estetrol-containing granules and drospirenone or a capsule comprising estetrol-containing granules and drospirenone. Optionally, the drospirenone may be comprised in the estetrol-containing granules. Alternatively, the drospirenone may be comprised in distinct granules, i.e. granules that do not comprise estetrol. The term “solid or semi-solid dosage unit” also encompasses capsules that contain a liquid, e.g. an oil, in which estetrol and drospirenone are dissolved or dispersed.

Tablets and equivalent solid and semi-solid dosage units can suitably contain materials such as binders (e.g. hydroxypropylmethyl cellulose, polyvinyl pyrrolidone (povidone, PVP), other cellulosic materials and starch), diluents (e.g. lactose (monohydrate) and other sugars, starch (e.g. maize starch), dicalcium phosphate and cellulosic materials), disintegrating agents (e.g. starch polymers and cellulosic materials (e.g. sodium starch glycolate) and lubricating agents (e.g., (magnesium) stearates and talc). These tablets and equivalent solid dosage units may be prepared by any suitable means, which have been described in detail in the art (e.g. Kaur, Int Res J Pharm, 2012). Non-limiting examples of processing methods of the estetrol and drospirenone when manufacturing the dosage unit include wet granulation, e.g. using an aqueous solution or an organic solution, direct compression, 3D printing, or by coating carrier particles with the estetrol and optionally with the drospirenone (either on the same carrier particles or distinct particles) using an organic or inorganic solvent.

Optionally, the excipient may be an active pharmaceutical ingredient excipient, binder excipient, carrier excipient, co-processed excipient, coating system excipient, controlled release excipient, diluent excipient, disintegrant excipient, dry powder inhalation excipient, effervescent system excipient, emulsifier excipient, lipid excipient, lubricant excipient, modified release excipient, penetration enhancer excipient, permeation enhancer excipient, pH modifier excipient, plasticiser excipient, preservative excipient, preservative excipient, solubiliser excipient, solvent excipient, sustained release excipient, sweetener excipient, taste making excipient, thickener excipient, viscosity modifier excipient, filler excipient, compaction excipient, dry granulation excipient, hot melt extrusion excipient, wet granulation excipient, rapid release agent excipient, increased bioavailability excipient, dispersion excipient, solubility enhancement excipient, stabilizer excipient, capsule filling excipient, or any combination hereof A skilled person is aware that use of such media and agents for pharmaceutical active substances is common practice and incorporation of these excipients is hence well known in the art. It is evident that all of the used ingredients should be non-toxic in the concentration contained in the final pharmaceutical composition or dosage unit and should not negatively interfere with the activity of the one or more pharmaceutically active ingredients, in the present context at least estetrol and drospirenone.

As described above, the composition, and hence the (oral) dosage unit may comprise one or more suitable excipients. The term “excipient” may be a “carrier” indicative for any solvent, diluent, buffer (including but not limited to neutral buffered saline, phosphate buffered saline, or optionally Tris-HCl, acetate or phosphate buffers), solubiliser (including but not limited to Tween 80 or Polysorbate 80), colloid, dispersion medium, vehicle, filler, chelating agent (including but not limited to EDTA or glutathione), amino acid, protein, disintegrant, binder, lubricant, wetting agent, stabiliser, emulsifier, sweetener, colorant, flavoring, aromatiser, thickener, any agent suitable to achieve a depot effect, coating, antifungal agent, any preservative (including but not limited to Thimerosal™ benzalkonium chloride, or benzyl alcohol), antioxidant (including but not limited to ascorbic acid, sodium metabisulfite), tonicity controlling agent, absorption delaying agent, adjuvant, bulking agent (including but not limited to lactose, mannitol) and any other ingredient that may influence any parameter or characteristic of the oral dosage unit subject of the invention. A skilled person understands that one or more excipients may be used in the composition or oral dosage unit on condition that the one or more excipient is compatible with the pharmaceutical ingredients (i.e. in the context of the present disclosure at least estetrol and drospirenone) and that a pharmaceutically acceptable formulation is obtained.

Optionally, the composition is comprised in a tablet comprising in addition to estetrol and drospirenone a filler, a superdisintegrant, a binder and disintegrant, a further binder, and a lubricant. In preferred embodiments, the composition is comprised in a tablet comprising estetrol and drospirenone, lactose, sodium starch glycolate, maize/corn starch, povidone, and magnesium stearate. Preferably, the composition is comprised in a tablet comprising estetrol monohydrate, drospirenone, lactose monohydrate, sodium starch glycolate type A, maize starch, povidone K30, and magnesium stearate. Optionally, the tablet is coated with a coating agent. In further optional embodiments, the coating agent comprises hypromellose, hydroxypropylcellulose, titanium dioxide, red iron oxide, hydrogenated cottonseed oil, and talc. By means of illustration and not limitation, a suitable coating agent is AquaPolish Pink 044.08 MS. A skilled person further appreciates that any excipients present in any dosage unit such as an oral dosage unit should adhere to pharmaceutical grade industry quality standards such as Ph. Eur. and USP-NF.

The (solid) oral dosage unit may be suitable or even specifically manufactured for sublingual, buccal, and/or sublabial administration. The dosage unit may be able to rapidly release the estetrol and drospirenone when contacted with an aqueous solvent such as saliva. Hence, in such embodiments the solid dosage unit is an orodispersible dosage unit which releases at least about 50%, preferably at least about 60%, more preferably at least about 70%, yet more preferably at least about 80%, most preferably more than about 80% of the estetrol and/or drospirenone within about 5 minutes, preferably within about 3 minutes, more preferably within about 2.5 minutes, more preferably within about 90 seconds, most preferably within about 90 seconds. The dosage unit may be an orodispersible dosage unit. In such embodiments, the dosage unit rapidly disintegrates in the oral cavity when it comes into contact with saliva and to disperse the estetrol and/or drospirenone into the saliva so it may be absorbed through the mucosal lining of the oral cavity. A skilled person is aware of methods to determine the release rate of a pharmaceutically active ingredient such as estetrol and drospirenone from a dosage unit. Non-limiting standardized tests generally accepted in the field include the disintegration test according to Ph. Eur. 2.9.1 (“Disintegration of tablets and capsules”) and USP <701> (“Disintegration”), for example using water as the disintegration medium.

The term “sublingual” as used herein refers to the pharmacological route of administration by which the estetrol and/or drospirenone (comprised in the dosage unit) diffuse into the blood through tissues under the tongue.

The term “buccal” as used herein refers to the pharmacological route of administration by which the estetrol and/or drospirenone (comprised in the dosage unit) diffuse into the blood through tissues of the buccal vestibule, the area inside the mouth between the lining of cheek (the buccal mucosa) and the teeth/gums.

The term “sublabial” as used herein refers to the pharmacological route of administration by which the estetrol and/or drospirenone (comprised in the dosage unit) are placed between the lip and the gingiva.

In certain embodiments, the estetrol and drospirenone are formulated into a solid dosage unit, including but not limited to hard capsules, soft capsules, tablets, coated tablets such as lacquered tablets or sugar-coated tablets, granules, aqueous or oily solutions, syrups, emulsions, suspensions, ointments, pastes, lotions, gels, inhalants or suppositories. In embodiments wherein the effective amounts of estetrol and drospirenone are administered by means of oral administration, the oral dosage unit according to the invention is preferably a solid or semi-solid dosage unit such as tablets, capsules, cachets, pellets, pills, powders and granules. The term “solid or semi-solid dosage unit” also encompasses capsules that contain a liquid, e.g. an oil, in which the present estetrol and drospirenone are dissolved or dispersed. Tablets and equivalent solid and semi-solid dosage units can suitably contain materials such as binders (e.g. hydroxypropylmethyl cellulose, polyvinyl pyrrolidone, other cellulosic materials and starch), diluents (e.g. lactose and other sugars, starch, dicalcium phosphate and cellulosic materials), disintegrating agents (e.g. starch polymers and cellulosic materials) and lubricating agents (e.g., stearates and talc). These tablets and equivalent solid dosage units may be prepared by any suitable means, which have been described in detail in the art (e.g. Kaur, Int Res J Pharm, 2012). Non-limiting examples of processing estetrol and drospirenone when manufacturing the dosage unit include wet granulation, e.g. using an aqueous solution or an organic solution, direct compression, 3D printing, or by coating carrier particles with the estetrol and drospirenone using an organic or inorganic solvent.

By means of illustration and not limitation, an oral dosage unit comprising the composition subject of the present disclosure may be manufactured by a process involving wet granulation. A skilled person appreciates that a wet granulation process may suitable comprise the successive steps of: dispensing and sieving of the active ingredient(s) and excipients, blending the sieved materials in a processor, granulation, screening (i.e. further sieving) of the granules, and one or more blending steps of the sieved granules with one or more further excipients. Afterwards, if desired in view of the final dosage unit the granules may be compressed into for example a tablet, optionally involving a coating step of said tablet.

The estetrol may be comprised in the composition and final dosage unit as particles. Optionally, the estetrol particles have a D(10) from about 0.5 μm to about 10 μm, preferably from about 1 μm to about 5 μm, more preferably of from about 1.5 μm to about 2.5 μm. Optionally, the estetrol particles have a D(50) of less than 20 μm or preferably less than 12 μm, more preferably from about 5 μm to about 15 μm, preferably from about 6 μm to about 12 μm, more preferably from about 7 μm to about 11 μm, most preferably from about 8 μm to about 12 μm. Optionally, the estetrol particles have a D(90) from about 15 μm to about 50 μm, preferably from about 20 μm to about 30 μm, more preferably from about 22 μm to about 28 μm. Optionally, the estetrol particles are further granulated into larger granulates. In certain embodiments, estetrol is comprised in the composition as a multitude of larger granulates that have a volume median diameter from about 100 μm to about 4000 μm, preferably from about 200 μm to about 1000 μm, more preferably from about 200 μm to about 600 μm.

A multitude of measurement techniques are available for determining particle-size distribution values and include sieve analysis, air elutriation analysis, photo analysis, optical counting, electro resistance counting, sedimentation, laser diffraction, laser obscuration, time of transition, acoustic spectroscopy, ultrasound attenuation microscopy, by means of a cascade impactor, or any combination thereof. Unless explicitly mentioned otherwise, the particle-size distribution values of the present disclosure are obtained by laser diffraction analysis. Laser diffraction analysis, interchangeably annotated in the art by laser diffraction spectroscopy, is a particle measurement technology based on interpretation of laser diffraction patterns passed through an object. Laser diffraction is capable to measure the geometrical dimensions of a particle. Laser diffraction protocols have been described in detail in the art on numerous occasions (e.g. as reviewed in detail in a context of particle analysis in Eshel et al., Soil Science Society of America Journal, 2004).

The endometriosis-associated or endometriosis-induced pain may be the result of endometrial tissue engrafts outside of the uterine cavity in the pelvis, optionally concurrent with adenomyosis and uterine fibroids. “Pelvis” as used herein is to be interpreted according to the commonly accepted definition in the art, i.e. the lower part of the torso between abdomen and thighs. The pelvis contains an embedded skeleton, commonly annotated as the pelvic skeleton. Similarly, a skilled person readily appreciates that the uterine cavity relates to the inside of the uterus. The uterine cavity is formed by the internal portion of the uterus body and flanked by the fallopian tubes (the crest of the internal orifice of the uterus enabling communication with the canal of the cervix). Optionally, the pain is resulting from endometriosis in distant (extraperitoneal) sites including but not limited to the colon, kidney, liver, pancreas, and lungs. Optionally, the endometrial tissue grafts may lead to ovarian lesions, peritoneal lesions, rectovaginal lesions, or any combination thereof, as described further below. Optionally, the pain is resulting from endometrial tissue engrafts located on the ovaries, fallopian tubes, tissues that hold the uterus in place (ligaments), outer surface of the uterus, vagina, cervix, vulva, bowel, bladder, rectum, or any combination thereof.

Optionally, the pain is a pain selected from the group consisting of: chronic pelvic pain, pelvic pain such as lower abdominal pain and/or low back pain, defecation pain and sexual intercourse pain, or pain caused by adhesion of endometrium in the Douglas pouch. “Chronic pelvic pain” as used herein is defined as pain in the pelvic area that lasts for six months or longer. The chronic pelvic pain may be continuous or periodical, optionally cyclical, and this is not limiting for the scope of the invention. As referred to herein, the “Douglas pouch”, interchangeably indicated by the terms “rectouterine pouch”, “rectovaginal pouch”, or “cul-de-sac” refers to the extension of the peritoneum between the rectum and the posterior wall of the uterus. It is commonly known that the Douglas pouch is the deepest point of the peritoneal cavity.

The composition for use, the use, and the methods described herein each result in a significant improvement of pain associated with, or induced by, endometriosis, particularly in subject characterized by a VAS score of at least about 40 mm, at least about 50 mm, at least about 60 mm, at least about 70 mm where a clear improvement can be observed when compared to hormonal treatment strategies described in the art. Methods to assess this improvement of pain are not limiting for the invention. Optionally, the improvement is measured by means of the Clinician Global Impressions-Improvement Scale (CGI-I Scale) as has been described in detail in the art and is therefore well known to a skilled person (e.g. Busner and Targum, Psychiatry, 2007). The CGI-Improvement (CGI-I) is assessed each time the patient is seen after medication has been initiated, by a clinician comparing the patient's overall clinical condition to the one week period just prior to the initiation of medication use (the so-called baseline visit). The following query is rated on a seven-point scale: “Compared to the patient's condition at admission to the project [prior to medication initiation], this patient's condition is:
1=very much improved since the initiation of treatment;
2=much improved;
3=minimally improved;
4=no change from baseline (the initiation of treatment);
5=minimally worse;
6=much worse;
7=very much worse since the initiation of treatment.”

In a preferred embodiment, at least about 10%, preferably at least about 15%, more preferably at least about 20%, more preferably of at least about 28%, or most preferably of at least about 28.9%, or about 28.9% of the patients show an improvement when compared to the situation prior to the start of administration of the composition. Alternatively, the pain improvement may be “minimally improved” corresponding to a CGI-I of 3, preferably “much improved” corresponding to a CGI-I of 2, most preferably “very much improved” corresponding to a CGI-I of 1 since the initiation of treatment. The CGI-I Scale scoring is considerably improved when compared to known treatment strategies, such as but not limited to a preparation comprising ethinylestradiol and drospirenone, more particularly 20 μg ethinylestradiol and 3 mg drospirenone in a fixed dose combination tablet administered in a flexible extended-cycle oral contraceptive regimen. Optionally, a CGI-I Scale improvement corresponding to “minimally improved or more” (CGI-I score 1 to 3) is obtained in at least about 75% of the subjects, preferably in at least about 77.5% of the subjects, more preferably in at least about 80% of the subjects, most preferably in at least about 82% of the subjects.

Alternatively, the improvement may be measured by means of the Patient Global Impressions-Improvement Scale (PGI-I Scale) which is known to a skilled person (e.g. Viktrup et al., BMC urol, 2012). The improvement may result in an improvement graded “marginally satisfied or more” (PGI-I score 1 to 3) in at least about 30%, preferably at least about 40%, more preferably at least about 50%, more preferably of at least about 60%, more preferably of at least about 70%, yet more preferably of at least about 75%, most preferably at least about 75.5% of the patients as compared to the situation prior to the start of administration of the composition. The improvement may result in an improvement graded “significant satisfied or more” (PGI-I score 1 and 2) in at least about 10%, preferably of at least about 20%, more preferably of at least about 24% of the patients. Alternatively, the pain improvement may be “a little better” corresponding to a PGI-I of 3, preferably “much better” corresponding to a PGI-I of 2, most preferably “very much better” corresponding to a PGI-I of 1 since the initiation of treatment. The PGI-I Scale scoring is considerably improved when compared to known treatment strategies, such as but not limited to a preparation comprising ethinylestradiol and drospirenone, more particularly 20 μg ethinylestradiol and 3 mg drospirenone in a fixed dose combination tablet administered in a flexible extended-cycle oral contraceptive regimen. Optionally, a PGI-I Scale improvement corresponding to “marginally satisfied or more” (PGI-I score 1 to 3) is obtained in at least about 67.5% of the subjects, preferably in at least about 70% of the subjects, more preferably in about 72.5% of the subjects, most preferably in at least about 75% of the subjects.

Optionally, the use of the compositions and methods described herein may result in a marked improvement in life quality of a subject when compared to a timepoint before start of the usage (i.e. treatment). Multiple testing methods have been described in the art that evaluate the quality of life of a subject, for example the Quality of Life Scale (QOLS) (Burckhardt and Anderson, Health Qual Life Outcomes, 2003). Hence, the quality of life as measured by the QOLS of a subject may be improved by at least 10%, preferably by at least 20%, preferably by at least 30%, preferably by at least 40%, preferably by at least 50% when compared to the QOLS score of said subject prior to the start of administration of the composition.

The composition for use, the use, and the methods described herein are characterised by a high responder proportion (throughout a general population). Optionally, the composition for use, the use, and the methods result in a responder proportion of at least about 40%, preferably at least about 50%, more preferably at least about 60% or about 70%. The responder proportion is considerably improved when compared to known treatment strategies, such as but not limited to a preparation comprising ethinylestradiol and drospirenone, more particularly 20 μg ethinylestradiol and 3 mg drospirenone in a fixed dose combination tablet administered in a flexible extended-cycle oral contraceptive regimen.

Optionally, the dosage unit is administered to a subject by means of a continuous administration schedule. The terms “continuous” and “continuously” as used herein, means that the dosage units are administered at relatively regular intervals, with no (therapeutically) significant interruptions. Naturally, minor interruptions may occur that do not affect the overall effectiveness of the present method, and indeed such aberrations are encompassed by the present invention. Preferably and more arithmetically, the administration regimen is deemed to be continuous if the longest interval between two subsequent administrations is not more than 3.5 times as long as the average interval. Even more preferably said longest interval is not more than 2.5 times, most preferably not more than 1.5 times as long as the average interval. By means of illustration and not limitation, the treatment strategies and method of treatments described herein preferably employ continuous administration of the estetrol and drospirenone during a period of at least 10 days, preferably of at least 20 days.

Preferably, the pharmaceutical composition described herein is used in cycles of from 21 to 28 daily administrations (i.e. a cyclical administration schedule), such as administration of 21 to 28 daily active dosage units. The pharmaceutical composition described herein may be used in cycles of 21, 22, 23, 24, 25, 26, 27, or 28 daily administrations by means of administration of a corresponding amount of daily active dosage units. The cycle preferably further comprises an administration-free interval of 7, 6, 5, or 4 days. Preferably, the cycle comprises an administration-free interval of 4 days.

As indicated above and supported by the example below, use of the composition results in a significant decrease in VAS score from prior to administration to after the third administration cycle, preferably from prior to administration to after the second administration cycle. It is understood that one administration cycle corresponds to one period of daily administrations as described above complemented with an administration-free interval as described above. Optionally, the use results in a decrease in VAS score of at least 10%, preferably at least 20%, more preferably at least 30%, more preferably at least 40%, more preferably at least 50%, more preferably at least 60%, more preferably at least 70%, more preferably at least 80%, more preferably at least 90%, from prior to administration to after the third administration cycle. Preferably, the use results in a decrease in VAS score of at least 10%, preferably at least 20%, more preferably at least 30%, more preferably at least 40%, more preferably at least 50%, more preferably at least 60%, more preferably at least 70%, more preferably at least 80%, more preferably at least 90%, from prior to administration to after the second administration cycle. Optionally, the use results in a decrease in VAS score to such an extent that after the second administration cycle a VAS score of less than about 40 mm is obtained, preferably of less than about 30 mm, preferably of less than about 20 mm, most preferably of less than about 15 mm, or even less than about 10 mm after the third administration cycle, preferably after the second administration cycle. Optionally, the use results in a reduction in VAS score from prior to administration to after the third administration cycle of at least about 10 mm, preferably of at least about 20 mm, preferably of at least about 30 mm, preferably of at least about 40 mm, preferably of at least about 50 mm, preferably of at least about 60 mm, preferably of at least about 70 mm.

The decrease in VAS score as described above is maintained (i.e. kept at a stable score or a generally constant reduced score) upon further administration cycles, such as after the second or after the third administration cycle. The pain reduction, pain suppression effect of the use of the composition is thus a long-lasting effect without any diminishment in efficacy over time.

Optionally, the use of the composition results in improvement of at least one parameter selected from the group consisting of: the severity of Douglas induration, restriction of uterine mobility, pelvic tenderness, reduced size of ovarian chocolate cysts, and the number of ovarian chocolate cysts as assessed by TVUS or MRI. In preferred embodiments, each of these parameters are improved by use of the composition. Preferably, the severity of Douglas induration is improved by at least about 10%, preferably at least about 20%, preferably at least about 30%, preferably at least about 40%, preferably at least about 50%, preferably at least about 60%, preferably at least about 70%, preferably at least about 80%, preferably at least about 90%. “Induration” as used herein refers to the thickening and/or hardening of soft tissues of the body, in the context of the present disclosure the Douglas pouch. Preferably the major axis of ovarian chocolate cysts is reduced by at least about 10 mm, preferably at least about 20 mm, preferably at least about 24.88 mm. Preferably the minor axis of ovarian chocolate cysts is reduced by at least about 10 mm, preferably at least about 20 mm, preferably at least about 26.24 mm. Preferably the volume of ovarian chocolate cysts is reduced by at least about 10 cm³, preferably at least about 20 cm³, preferably at least about 30 cm³, preferably at least about 39.56 cm³.

In a preferred embodiment, the use of the composition results in a decrease of cancer antigen 125 (CA125). Preferably, CA125 serum levels are decreasing to below 35 U/ml. “CA125” may be interchangeably indicated in the art as “MUC-16” or “Mucin-16” and is encoded in humans by the MUC16 gene.

The composition for use, the use, and the methods described herein are characterised by a low incidence of Treatment Emergent Adverse Events (TEAEs) when compared to known treatment strategies in the art that rely on administration of an estrogen and drospirenone, more particularly 20 μg ethinylestradiol and 3 mg drospirenone in a fixed dose combination tablet administered in a flexible extended-cycle oral contraceptive regimen. It is to be appreciated that TEAEs are undesirable events caused by the use of a certain pharmaceutically active ingredient, a composition, or a dosage unit in a subject that were not present before administration thereof to said subject. Optionally, the TEAEs are selected from the group consisting of intermenstrual bleeding, headache, nausea, heavy menstrual bleeding, including any combination thereof. Preferably, the sum of all TEAEs is at least 10% lower, preferably at least 20% lower, preferably at least 30% lower, preferably at least 40% lower, preferably at least 50% lower when compared to known treatment strategies, such as a treatment relying on administration of an estrogen and drospirenone, more particularly 20 μg ethinylestradiol and 3 mg drospirenone in a fixed dose combination tablet administered in a flexible extended-cycle oral contraceptive regimen.

While drospirenone, and particularly from 2.5 mg to 3.5 mg of drospirenone, more particularly about 3 mg of drospirenone is highly preferred in the context of the present invention, other progestogenic components are also envisaged as alternatives. Examples thereof include without limitation: levonorgestrel, norgestimate, norethisterone, dydrogesterone, 3-beta-hydroxydesogestrel, 3-ketodesogestrel, 17-deacetylnorgestimate, 19-norprogesterone, acetoxypregnenolone, allylestrenol, amgestone, chlormadinone, cyproterone, demegestone, desogestrel, dienogest, dihydrogesterone, dimethisterone, ethisterone, ethynodiol diacetate, fluorogestone acetate, gastrinone, gestodene, gestrinone, hydroxymethylprogesterone, hydroxyprogesterone, lynestrenol, mecirogestone, medroxyprogesterone, megestrol, melengestrol, nomegestrol, norethindrone, norethynodrel, norgestrel (including d-norgestrel, and dl-norgestrel), norgestrienone, normethisterone, progesterone, quingestanol, (17 alpha)-17-hydroxy-11-methylene-19-norpregna-4,15-dien-20-yn-3-one, tibolone, trimegestone, algestone-acetophenide, nestorone, promegestone, 17-hydroxyprogesterone esters, 19-nor-17hydroxyprogesterone, 17alpha-ethynyltestosterone, 17alpha-ethynil-19-nortestosterone, d-17beta-acetoxy-13beta-ethyl-17alpha-ethynylgon-4-en-3-one oxime, 6beta, 7beta;15beta,16beta-dimethylene-3-oxo-17-pregna-4,9(11)-diene-21,17beta-carbolactone or tanaproget and precursors of these compounds that are capable of liberating these progestogens in vivo.

A further aspect of the invention is directed to packaging units comprising the dosage units described herein. The packaging units may comprise at least 14, preferably at least 21, even more preferably at least 28, containers for holding separately packaged and individually removable dosage units, wherein each container comprises at least one dosage unit comprising of from about 13.5 to about 16.5 mg of estetrol or estetrol monohydrate, preferably about 15 mg of estetrol monohydrate, and from about 2.5 mg to about 3.5 mg drospirenone, preferably about 3 mg of drospirenone. Preferably, the separately packaged and individually removable dosage units are oral dosage units. More preferably, each of the separately packaged and individually removable dosage units comprise from about 13.5 mg to about 16.5 mg of estetrol or estetrol monohydrate, preferably about 15 mg of estetrol monohydrate, and from about 2.5 mg to about 3.5 mg of drospirenone. Optionally, each of the additional containers for holding the dosage units comprising the estetrol and drospirenone are individually visually arranged to present a recommended order of administration.

The skilled person appreciates that within the scope of the present invention, each packaging unit, e.g. blister pack, may be numbered or otherwise marked. The packaging units may be provided in any suitable packaging means known in the art, non-limiting examples being troches, sachets, pouches, bottles, films, sprays, microcapsules, implants, rods or blister packs.

By means of illustration and not limitation, each packaging unit may be a sealed blister pack with a cardboard, paperboard, foil plastic backing and enclosed in a suitable cover. Also envisaged in any one of the aspects defined herein are packaging units such as bottles. The material of the bottle is not particularly limiting. In preferred embodiments, the bottle is a glass bottle characterised by a colour capable of reducing or preventing degradation of the contents of the bottle by e.g. UV light while maintaining a degree of transparency that allows for visual inspection of the contents of said bottle. Suitable colours include without limitation amber, cobalt, or vintage green.

In a particular embodiment of the invention the packaging unit comprises 28 containers or a multiple of 28 containers, such as 2 to 12 multiples of 28 containers.

The packaging unit of the contraceptive kit disclosed herein may be a “compliance package”. As known from the art, “compliance packages” are packaging units of variable sizes and formats that, next to providing a suitable storage means for one or more medicaments, aim to provide assistance and/or guidance to a subject to comply with the intended periodical administration (Peck Gossel, Packaging the Pill, Manifesting Medicine: Bodies and Machines, New York: Taylor & Francis, 1999). As a non-limiting example, the packaging unit may be provided with numerical indications and/or symbols that allow the subject to keep track of for example said subject's menstrual cycle. In an alternative non-limiting example, the packaging unit may comprise means to send an electronic signal to a subject when a predefined time of administration (i.e. a certain day) is reached and the dosage unit for that time point is still contained in the packaging unit. In those examples, the electronic signal may be sent to a data storage means and/or sent to a user-defined electronic device, smartphones and smart wear begin illustrative examples hereof. In certain embodiments, distinct portions of the packaging unit(s) provide a different sensory trigger to a subject, non-limiting examples being distinct colors or roughness.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples. The following specific experimental examples are provided in support of the claimed invention but are not to be seen as limiting the scope of the invention.

### EXAMPLES

### Example 1. An Open Label, Parallel Group Study to Explore Pharmacodynamics, Pharmacokinetics and Safety During 3 Cyclic Treatments of a 15 mg E4 Monohydrate/3 mg DRSP Tablet in Japanese Endometriosis Patients

### Study Objective

To explore pain-relieving effect, pharmacodynamics, pharmacokinetics and safety on the blood coagulation/fibrinolytic system and endocrine system during three treatment cycles of a 15 mg E4 monohydrate/3 mg DRSP tablet (hereafter “E4/DRSP”) to patients with endometriosis for 24 days every day, followed by a 4-day placebo medication period for 28 days.

### Study Design

Multi-center, open-label, randomized, parallel group
Phase, Type of Study
Phase II, Clinical Pharmacology

### Eligibility Criteria

### Criteria for Inclusion

1. Diagnosed as endometriosis by laparotomy/laparoscopy, and/or as having ovarian chocolate cysts by TVUS or MRI
2. Aged ≥20 and ≤50 years
3. Pelvic pain (defined as a VAS score ≥40 mm) during the baseline observation phase
4. Regular menstrual cycles (25-38 days) during the baseline observation phase
5. BMI<30 kg/m2
6. Fully understood the content of this trial and agreed in writing to participate in the study

### Criteria for Exclusion

1. Undiagnosed abnormal vaginal bleeding 6 months before the screening tests
2. Aged ≥40 years with endometriomas for which the largest diameter was >10 cm
3. Endometriomas containing solid components
4. Had undergone surgical treatment for endometriosis by cyst puncture (such as transvaginal alcohol fixation), laparotomy, or laparoscopy (laparoscopy) within 2 months prior to the screening tests
5. Had not improved endometriosis symptoms (moderate or severe pelvic pain) with the oral contraceptive or hormone preparations containing progestin
6. Had been judged by the investigator to have priority for surgical treatment of organic diseases
7. Presence or history malignant tumors (e.g. cervical intraepithelial neoplasia, cervical cancer, breast cancer). Non-melanoma skin cancer is acceptable
8. Presence or history of deep vein thrombosis, thrombophlebitis (excluding superficial), pulmonary embolism, cerebrovascular disorder, coronary artery disease, etc.
9. Aged ≥35 years who smokes ≥15 cigarettes/day
10. Migraine with auras (flash scotoma, star-shaped flash, etc.)
11. Presence of valvular heart disease associated with pulmonary hypertension or atrial fibrillation, or valvular heart disease with a history of subacute bacterial endocarditis
12. Diabetes associated with vascular lesions such as diabetic nephropathy and diabetic retinopathy
13. Thrombotic predisposition (e.g. antithrombin, protein S and protein C deficiencies)
14. Phospholipid antibody syndrome (e.g. antiphospholipid antibody positive or unknown systemic lupus erythematosus)
15. Had been scheduled for surgery within 4 weeks after obtaining consent or had undergone surgery within 2 weeks before obtaining consent
16. Presence of severe liver impairment (e.g. acute viral hepatitis, severe cirrhosis, etc.)
17. Presence of liver tumor
18. Presence of severe (GFR<60 mL/min/1.73 m2) or acute kidney impairment
19. History or presence of heart disease such as uncomplicated valvular heart disease
20. Hypertension [systolic blood pressure ≥140 mmHg and/or diastolic blood pressure ≥90 mmHg]
21. Ear sclerosis patients
22. History of jaundice, persistent pruritus, or herpes during pregnancy
23. Pregnant women or women who may be pregnant
24. Gave birth or miscarried in the second trimester within 6 weeks prior to enrolment
25. Women who are breastfeeding
26. Hypersensitivity against active ingredients of the study drug
27. Has been contraindicated for Yaz Flex combination tablets
28. History of discontinuation of sex steroid hormone treatment due to adverse events or hypersensitivity
29. Has been receiving drugs or their derivatives that are thought to affect sex hormone secretion
30. Had taken the following drugs 1 month before the screening:
   Hormone preparations containing progestin, estrogen, low dose birth control pills, fixed dose combination of progestin and estrogen
   GnRH agonist/antagonist, testosterone derivatives
   Estrogen antagonists, aromatase inhibitors
   Herbal medicines to treat dysmenorrhea, endometritis, menstrual pain
   Anticoagulants
   Hypercholesterolemia drugs, antidiabetic drugs (including insulin), drugs that affect serum potassium levels (ACE inhibitors, ARBs, potassium-sparing diuretics, aldosterone antagonists)
   Minor tranquilizers, antispasmodics
31. Current use or use of the following drugs within 1 month prior to enrolment
   CYP3A4 inducers (e.g. carbamazepine, phenobarbital, phenytoin, rifabutin, rifampicin, St. John's wort, etc.)
   CYP3A4 inhibitors (e.g. cobicistat, indinavir, itraconazole, ritonavir, telaprevir, voriconazole, clarithromycin, nelfinavir, saquinavir, grapefruit juice, etc.)
   HIV protease inhibitors, HCV protease inhibitors, non-nucleoside reverse transcriptase inhibitors
32. Regular use of analgesics for medical reasons other than relief of endometrial pain during the study (occasional use will be permitted; prophylaxis is not allowed)
33. Had participated in another trial within 1 month before the screening tests or received other investigational drugs within 3 months before the screening test. Had participated in clinical trials of other oral contraceptives containing an approved active ingredient in Japan and completed those trials within 2 months before the screening tests
34. Patients who wish to become pregnant or do not agree to prohibit sexual intercourse or contraception
*) During the study period
*) Use of barrier contraceptive devices approved or certified in Japan (latex condoms or contraceptive pessary for men)
35. Judged by the investigator to be ineligible.

### Number of Subjects

Eighty (80) subjects in total: 40 subjects in the E4/DRSP or the Yaz Flex group each.

[Note] Since this trial was an exploratory clinical pharmacology study, it was set with reference to past clinical study results. Based on the results of a Phase III study of Yaz Flex combination tablets in patients with endometriosis (change of VAS in the most advanced pelvic pain—lower abdominal pain/low back pain—from the previous observation period, assuming that the effect size (effect size) was 0.5 and the dropout rate was 15%, the number of cases that kept a power of 80% was estimated.

### Dose, Route of Administration and Duration of Treatment

### Investigational Group:

E4 monohydrate 15 mg/DRSP 3 mg, fixed dose combination tablet

Oral administration for 24 days and placebo administration for 4 days immediately after were given for 28 days with 3 cycles (84 days).

### Reference Group

Yaz Flex (EE 20 μg/DRSP 3 mg, fixed dose combination tablet)

Administered continuously until day 24, regardless of bleeding. If bleeding (including spotting) was observed for 3 consecutive days after the 25th day, discontinue the drug for 4 days. Continuous dosing was started regardless of whether bleeding has ended or was continuing. The drug administration period was implemented for 84 days.

### Excluded Medications and Dietary Products

### Criteria for Prohibited Medications

Hormone preparations containing progestin and/or estrogen, low dose OC, fixed dose combination of progestin and estrogen
GnRH agonists/antagonists, testosterone derivatives, estrogen antagonists, aromatase inhibitors
Drugs or their derivatives that are thought to affect sex hormone secretion
Minor tranquilizers, antispasmodics
CYP3A4 inducers (e.g. carbamazepine, phenobarbital, phenytoin, rifabutin, rifampicin, St. John's wort, etc.)
CYP3A4 inhibitors (e.g. cobicistat, indinavir, itraconazole, ritonavir, telaprevir, voriconazole, clarithromycin, nelfinavir, saquinavir, grapefruit juice, etc.)
Herbal medicines to treat dysmenorrhea, endometritis, menstrual pain
Anticoagulants
Hypercholesterolemia drugs, antidiabetic drugs (including insulin), drugs that affect serum potassium levels (ACE inhibitors, ARBs, potassium-sparing diuretics, aldosterone antagonists)
HIV protease inhibitors
HCV protease inhibitors
Non-nucleoside reverse transcriptase inhibitors
Other investigational drugs other than the investigational drug used in this trial

### Criteria for Prohibited Treatments

Cyst puncture (such as transvaginal alcohol fixation), laparotomy, or laparoscopic treatment or examination

### Use of Analgesics

Occasional use of analgesics for medical reasons (intolerable pain associated with endometriosis and adverse events) was permitted during the study: Loxoprofen tablet or granules (60 mg per time, ≤3 times in a day), Ibuprofen tablet or granules (200 mg per time, ≤3 times in a day)
Any NSAIDs other than Loxoprofen and Ibuprofen was not allowed during the study, and was withdrawn before visit 1 if applicable

### Main Criteria for Evaluation and Analyses

### 1. Severest Pelvic Pain

Visual analog scale (VAS) collected by a patient diary

### 2. Gynecological Examination

Douglas induration, restriction of uterine mobility, pelvic tenderness

### 3. Hemostasis and Related Parameters

Protein S (total activity, antigen (total), specific activity, antigen (free)), free TFPI antigen, Antithrombin activity, APC sensitivity ratio (APTT-based, ETP-based), D-dimer, Fibrinogen, Factor V/VII/X, Prothrombin time, Protein C, Plasminogen, tPA-PAI-1complex, Prothrombin Fragment 1+2, soluble Fibrin monomer complex, APTT, SHBG

### 4. Endocrine Parameters

Estradiol, Progesterone, LH, FSH

### 5. Safety

Adverse events, clinical laboratory tests, body weight, vital signs (blood pressure, pulse, temperature), 12-lead ECG, physical examination

### 6. Pharmacokinetics

Plasma concentration of E4, EE and DRSP

### Main Statistical Considerations

### 1. Severest Pelvic Pain (VAS): Per Protocol Set Population

Descriptive statistics (number of subjects, arithmetic mean, standard deviation, % coefficient of variation, quartile, minimum and maximum) was estimated to summarize the change from baseline of the VAS by stratification factors, by visits and by groups. ANOVA was also performed with stratification factors as a fixed effect. Within the framework of ANOVA, LS means and two-sided 95% confidence intervals were inferred for the values of change from baseline by visit and by group.

2. Pain Score Associated with Endometriosis and Dysmenorrhea Score: Per Protocol Set Population

Descriptive statistics was used for the change from the baseline of the “endometriosis pain score and dysmenorrhea score” during the treatments by group and by visit.

### 3. Gynecological Examination

Descriptive statics for continuous variables and frequency for categorical data were estimated, respectively.

### 4. Endocrine-Related Parameters

Descriptive statistics were used for the change from the baseline of the absolute and relative values by group and by visit.

### 5. Hemostasis and Related Parameters: Per Protocol Set Population

Descriptive statistics were used to summarize the change from baseline (observed and relative %) of each parameter by visit and by group. ANOVA was performed to estimate LS means and two-sided 95% confidence interval for the change from baseline (relative %) by visit and by group. The same ANOVA analysis was conducted for differences of LSmeans (E4/DRSP−Yaz) and two-sided 95% confidence intervals using a model considering an interaction term of treat-by-visit.

### 6. Safety: Safety Population

Descriptive statistics were used to summarize the continuous variables (clinical laboratory tests, blood pressure, pulse, temperature, body weight, 12-lead ECG) by visit and by group. Frequency tables giving number and percentage of subjects within each category were used in summarizing categorical (AE) variables. The latest version of MedDRA will be used.

### 7. Pharmacokinetics: Safety Population

Descriptive statistics were used to summarize the serum or plasma concentrations of E4, EE and DRSP by group and by visit. In addition, non-linear mixed effect model was applied to explore the exposure response relationship if applicable.

### Result Summary

### 1. Efficacy on Endometriosis Associated Pain

Both E4/DRSP and Yaz Flex were found to similarly diminish the values of VAS grading the most sever pelvic pain (lower abdominal and back pain) in Per Protocol Analysis Set (PPS) and Full Analysis Set (FAS) population. The change from baseline of the VAS value basically kept stable after the 2nd cyclic or 56-day flex administration, and resulted in −32.48±19.575 (mean±standard deviation, ditto) mm and −33.93±27.024 mm at the efficacy assessment period (EAP), i.e. 3rd cyclic or 84-day flex administration in E4/DRSP and Yaz Flex groups, respectively.

Numeric rating scale (NRS) also suggested that any types of pains associated with endometriosis be comparably improved between E4/DRSP and Yaz Flex groups through the treatment periods.

Dysmenorrhea scores, consisting of dysmenorrhea symptoms severity and analgesics use domains, were also reduced after the 2nd/and 3rd cyclic or 56- and 84-day administration in both groups, however a marginally larger improvement scores were observed in Yaz Flex group. Dysmenorrhea symptoms severity and analgesics use showed similar profile to dysmenorrhea scores. The use of analgesics was reduced at visit 5 and visit 6 in both E4/DRSP and Yaz Flex groups, however a bit larger improvement was achieved in Yaz Flex group.

Gynaecological examination showed the marginal improvement in the severity of Douglas induration, restriction of uterine mobility and pelvic tenderness, and size and number of ovarian chocolate cysts.

In the clinical global impressions—improvement (measured on the CGI-I scale) following 3 cyclic or 84-day flex administration, frequency of “improved or more” (CGI-I score 1 and 2) was 28.9% (13/45 subjects) in E4/DRSP group and 40% (16/40 subjects) in Yaz Flex. Similarly, E4/DRSP and Yaz Flex rated the frequencies of “marginally improved or more” (CGI-I score 1 to 3) as 82.2% (37/45 subjects) and 72.5% (29/40 subjects), respectively. In addition, the patient global impressions-improvement (PGI-I) found that 24% of subjects (11/45) and 75.5% of subjects (34/45) in E4/DRSP group graded “significantly satisfied or more” (PGI-I score 1 and 2) and “marginally satisfied or more” (PGI-I score 1 to 3), respectively. Similar grades were achieved in Yaz Flex group, which were estimated to be 32.5% (13/40 subjects) for “significantly satisfied or more” (PGI-I score 1 and 2) and 65.0% (26/40 subjects) for “marginally satisfied or more” (PGI-I score 1 to 3).

### 2. Effect on Blood Coagulation—Fibrinolysis System

Both Forest plot and ANOVA suggested that E4/DRSP gave very limited influence on blood coagulation-fibrinolysis system as compared to Yaz Flex, looking into the baseline variabilities of haemostatic parameters. Set or results can be summarized as in the tables 1 to 3 as below;

TABLE 1
Haemostatic parameters with statistical significance
only in Yaz Flex group (change from baseline, %)

| Parameters | E4/DRSP | Yaz Flex |
|---|---|---|
| D-dimer | 16.457% | 75.927% |
| PT (sec) | −1.59% | −4.91% |
| PT-INR | −1.561% | −4.779% |
| Soluble fibrin monomer | 1.06% | 8.84% |
| Protein S, antigen | 0.163% | −16.554% |
| Factor V | −1.7% | −8.9% |
| Protein C | 3.0% | 13.7% |
| Prothrombin Fragment F1 + 2 | 1.3% | 47.6% |

(p < 0.05)

TABLE 2
Haemostatic parameters with statistical significance in
E4/DRSP and Yaz Flex groups (change from baseline, %)

| Parameters | E4/DRSP | Yaz Flex |
|---|---|---|
| Fibrinogen | 11.2% | 15.2% |
| APTT (sec) | −8.00% | −11.21% |
| APTT (controlled) | −0.63% | −0.73% |
| Protein S (total activity) | 8.707% | −10.992% |
| Protein S (specific activity) | 8.987% | 6.728% |
| Protein S (free antigen) | 7.8% | −13.6% |
| Free TFPI (antigen) | −21.1% | −47.2% |
| APCsr (APTT-based) | −1.650% | −3.462% |
| APCsr (ETP-based) | 41.544% | 155.624% |
| Factor VII | 14.2% | 53.0% |
| Factor X | 17.4% | 32.1% |
| Plasminogen | 14.1% | 36.0% |
| SHBG | 63.12% | 210.40% |

(p < 0.05)

TABLE 3
Hemostatic parameters with statistical significance
in group difference of change from baseline
(%) between E4/DRSP and Yaz Flex groups

| | Group difference (lower, upper | |
|---|---|---|
| Parameters | confidence interval - 95%) | |
| D-dimer | −59.470 | (−115.442, −3.498) |
| PT (sec) | 3.32 | (0.88, 5.76) |
| PT-INR | 3.217 | (0.828, 5.607) |
| APTT (sec) | 3.21 | (0.62, 5.79) |
| Protein S (total activity) | 19.699 | (13.995, 25.402) |
| Protein S (antigen) | 16.717 | (11.892, 21.543) |
| Protein S, free (antigen) | 21.3 | (14.3, 28.4) |
| Free, TFPI (antigen) | 26.1 | (20.7, 31.6) |
| APCsr (APTT-based) | 1.812 | (0.417, 3.207) |
| APCsr (ETP-based) | −114.081 | (−151.250, −76.911) |
| Factor V | 7.2 | (2.9, 11.6) |
| Factor VII | −38.8 | (−48.5, −29.2) |
| Factor X | −14.7 | (−21.2, −8.1) |
| Protein C | −10.7 | (−16.8, −4.5) |
| Plasminogen | −22.0 | (−29.6, −14.4) |
| Prothrombin F1 + 2 | −46.3 | (−67.5, −25.1) |
| SHBG | −147.28 | (−178.26, −116.31) |

### 3. Pharmacokinetics

It was observed that higher drug concentrations were measured for E4 (E4/DRSP group), EE (Yaz Flex group) and DRSP (E4/DRSP and Yaz Flex groups) up to 2 hrs post-dose, which then gradually decreased with time. Non-linear mixed model analysis was applied to develop a two-compartment model with the first-order absorption and elimination for both E4 and DRSP. There seemed to be marginal relationship between CL on E4 and body size such as BMI, however no statistical significance was confirmed, unlikely suggesting potential subject covariates including DRSP. No non-linear mixed model was developed for EE.

### 4. Safety

Treatment emergent adverse events (TEAEs) were reported to be 88.9% (40/45 subjects) in E4/DRSP group and 92.7% (38/41 subjects) in Yaz Flex group. Of which, causal relationship could not be ruled out for TEAEs of 77.8% (35/45 subjects) and 90.2% (37/41 subjects) in E4/DRSP and Yaz Flex groups, respectively. No serious adverse event (SAE) was reported for E4/DRSP group, however one SAE, i.e. deep vein thrombosis was reported in Yaz Flex group. In Yaz Flex group, one severe TEAE, thrombosed haemorrhoids was observed, however there were no reports in E4/DRSP group. No sever TEAEs with causal relationship were reported in both treatment groups. One subject had immature termination due to fatigue and loss of appetite in Yaz Flex group. In contrast, there were no TEAEs leading to early termination in E4/DRSP group.

In E4/DRSP group, it was reported that high incident TEAEs were intermenstrual bleeding (68.9%, 31/45 subjects), headache (17.8%, 8/45 subjects), abdominal discomfort/nausea/heavy menstrual bleeding (6.7%, 3/45 subjects each). Of which, TEAEs with causal relationship were intermenstrual bleeding (68.9%, 31/45 subjects), headache (6.7%, 3/45 subjects) and heavy menstrual bleeding (6.7%, 3/45 subjects). In Yaz Flex group, as high incident TEAEs, intermenstrual bleeding, headache, nausea and heavy menstrual bleeding were reported at 73.2% (30/41 subjects), 26.8% (11/41 subjects), 22.0% (9/41 subjects) and 12.2% (5/41 subjects), respectively. In 3/41 subjects (7.3%), fatigue, edema and nasopharyngitis appeared. Of which, TEAEs with causal relationship were intermenstrual bleeding 73.2%, nausea 22.0%, headache and heavy menstrual bleeding 9.8% respectively, and edema 7.3%.

Endometrial thicknesses were 4.35±2.340 mm (mean/sd, ditto) in E4/DRSP group and 3.73±1.666 mm in Yaz Flex group following 3 cyclical or flex administrations, which were comparably thinning.

Days of bleeding were counted as 30.4±14.58 days in E4/DRSP and 27.4 14.29 days in Yaz Flex. Both groups resulted in the similar bleeding events, which was evaluated to be 4.5±1.19 events in E4/DRSP and 3.4±1.39 events in Yaz Flex. The bleeding events lasted for 7.32±4.750 days in E4/DRSP and 8.65±5.137 days in Yaz flex. In E4/DRSP group, spotting events were reported to be 23.3% (251/1076 days), 15.1% (159/1056 days) and 10.4% (110/1056 days) at the 1st, 2nd and 3rd cycle treatments (excluding placebo medication periods), respectively. In contrast, the events in Yaz Flex group took place at the rate of 22.9% (255/1113 days), 10.6% (105/991 days) and 8.8% (74/840 days) for 28-, 56- and 84-day flex treatments. Similar results were obtained for the bleeding events as well; E4/DRSP—29.0% (312/1076 days) at cycle 1, 8.0% (85/1056 days) at cycle 2 and 10.0% (106/1056 days) at cycle 3, Yaz Flex—27.9% (310/1113 days) for 28-day flex dosing, 7.6% (75/991 days) for 56-day flex dosing and 4.5% (38/840 days) for 84-day flex. Number of genital bleeding events were smaller in Yaz Flex than in E4/DRSP during the hormone free interval, and mostly spotting events.

There were no remarkable findings on clinical laboratory tests (including hematological tests, biochemical tests, urine tests, physiological examination and body mass (height, weight, BMI) in both groups.

No abnormal changes were observed for ECG following visit 2 in E4/DRSP. In contrast, ECG examination following visit 2 found 3 events in 2 subjects (abnormal ECG, right bundle branch block, first-degree atrioventricular block) in Yaz Flex group, all of which were diagnosed with complications.

No abnormal shifts of QTc interval were observed for both E4/DRSP and Yaz Flex groups. In E4/DRSP group, QTcB (Bazett type correction) and QTcF (Fredericia type correction) were evaluated to be 409.7±42.29 msec and 406.6±42.92 msec at Visit 6/EOS. Similar measurements were obtained for Yaz Flex, i.e. 419.3±21.99 msec (QTcB) and 415.0±20.04 msec (QTcF).

Ad-Hoc Analysis Results after Data Base Locked

This analysis aimed at characterizing subject population where E4/DRSP FDC (Fixed Dose Combination) tablet would be able to provide clinical benefit, juxtaposing with EE/DRSP FDC (Yaz Flex) in Japanese endometriosis patients.

The most sever pelvic pain over the course of two different menstrual periods (pre-treatment period) was estimated to be ca. 70 mm VAS value as the median value across the E4/DRSP and Yaz Flex groups, which meant that 50% of randomized subjects suffered from the pain intensity that was corresponding to 70 mm or more.

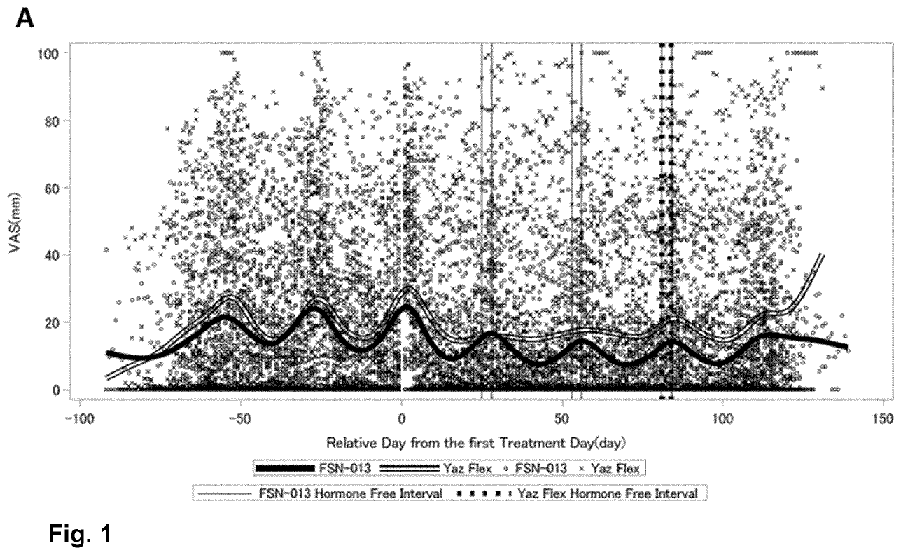

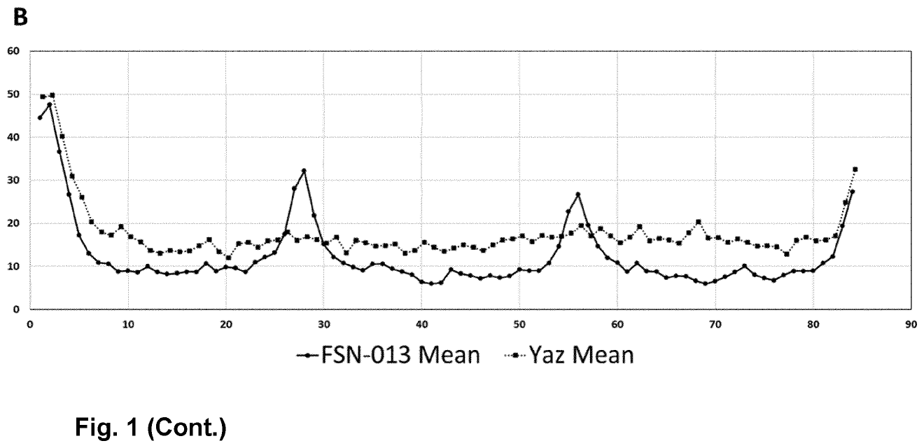

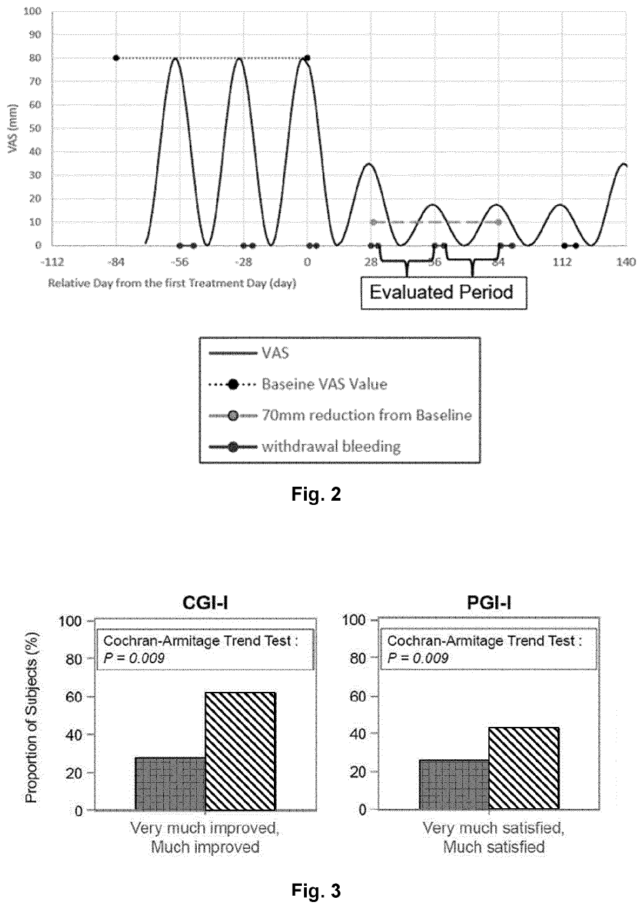

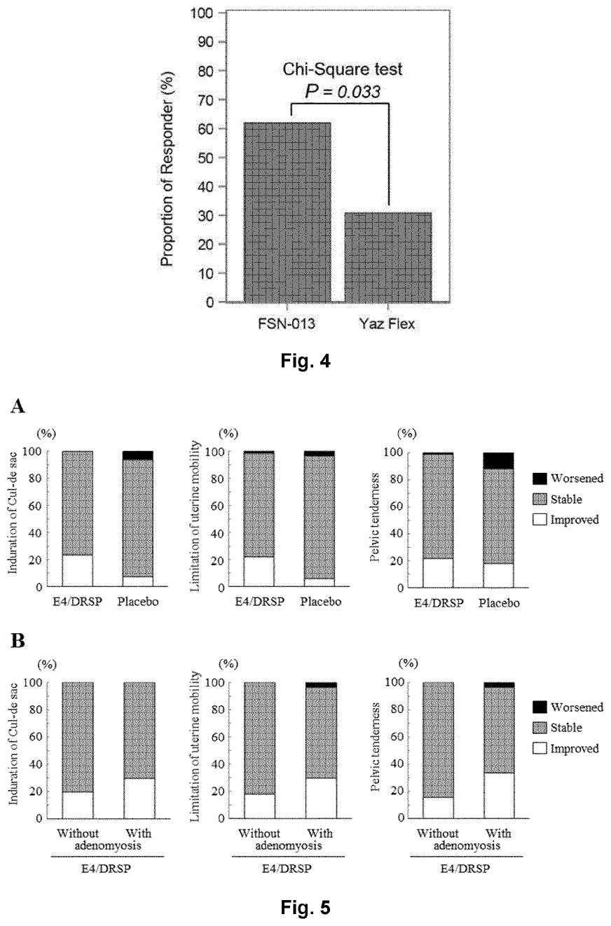

Set of the analyses consisted of three different parts. First, the individual pain intensities (VAS) over time profiles were depicted based on the daily VAS values, and spline function was applied to elucidate the VAS over time profile for each of drugs, i.e. E4/DRSP and Yaz Flex groups. As shown in FIG. 1, the spline curve profiles suggested that the pain intensities be differently suppressed during non-withdrawal bleeding periods between two drugs, and E4/DRSP attained more suppressive VAS reduction during the active dosing period as compared to Yaz Flex. As the second part, the remarkable VAS over time profiles were quantified according to the following definition—Proportion of days where the VAS values were reduced by 70 mm or more from the baseline during 80% or more of the evaluation periods (FIG. 2). The subjects who met the criteria is tentatively called as “responder” in this analysis report. Lastly, the key question was what clinical advantages were brought to the responder, and both CGI-I and PGI-I scores were investigated to link to the criteria. For this purpose, primary interest was that higher scores of CGI-I and/or PGI-I were observed in the responder. Cochran-Armitage test referred to a statistically significant trend in the responder (p=0.009), but not in the non-responder, which resulted in both clinical investigators and subjects marking much improved/satisfied or more scores in responder (diagonally striped, FIG. 3) when compared to non-responder (solid grey filled, FIG. 3). Finally, proportion of responder was compared between E4/DRSP and Yaz Flex. E4/DRSP group led to ca. 60% of the responder proportion, in contrast the proportion was ca. one-half, ca. 30% in Yaz Flex group (FIG. 4). The statistical significance was observed by a manner of Chi square test (p=0.033).

In summary, it was suggested that E4/DRSP would be able to reduce the pelvic pain during the non-menstrual period more than Yaz Flex, leading to improvement of CGI-I and PGI-I.

### Example 2. Efficacy and Safety of Estetrol Monohydrate 15 mg/Drospirenone 3 mg Combination (E4/DRSP) in a Cyclic Regimen for the Treatment of Endometriosis-Associated Pain and Objective Gynaecological Findings: A Multi-Center, Placebo-Controlled, Double-Blind, Randomized Study

### Material and Methods

### Study Design

A multi-center, randomized, double-blind, placebo-controlled, parallel-group design study was conducted in Japanese subjects with endometriosis. The objective was to investigate the potential of E4/DRSP to treat endometriosis-associated pelvic pain (EAPP) following a six-cycle treatment (24-days followed by a 4-day hormone-free interval per cycle) with E4/DRSP for 24 weeks.

### Subjects

Subjects aged ≥20 years and diagnosed with endometriosis were enrolled. The clinical diagnosis was determined by laparotomy/laparoscopy, transvaginal ultrasonography (TVUS), magnetic resonance imaging (existence of ovarian endometriomas) or gynecological examinations. Another critical eligibility criterion was ≥40 mm EAPP in the Visual Analog Scale (VAS) during the baseline observation period. Demographics and baseline characteristics are shown in Table 4.

TABLE 4
Demographics and baseline characteristics (FAS).

| | E4/DRSP | | Placebo | | Total | |
|---|---|---|---|---|---|---|
| Characteristic | n = 79 | | n = 83 | | n = 162 | |
| Age, years | 34.6 ± 6.89¹⁾ | | 34.8 ± 7.48 | | 34.7 ± 7.17 | |
| | [21-46] ²⁾ | | [21-48] | | [21-48] | |
| Height, cm | 159.44 ± 5.238 | | 160.45 ± 5.171 | | 159.96 ± 5.212 | |
| Weight, kg | 54.31 ± 6.444 | | 55.43 ± 7.836 | | 54.88 ± 7.191 | |
| BMI, kg/m² | 21.31 ± 2.369 | | 21.47 ± 2.805 | | 21.39 ± 2.595 | |
| Menstrual Cycle, days | 28.78 ± 2.672 | | 28.97 ± 2.863 | | 28.88 ± 2.764 | |
| Pelvic Pain (Lower Abdominal Pain, | 80.0 ± 14.93 | | 76.4 ± 14.70 | | 78.2 ± 14.87 | |
| Back Pain) VAS, mm | | | | | | |
| Number of Spotting or Bleeding, days | 6.77 ± 1.940 | | 6.75 ± 1.968 | | 6.76 ± 1.948 | |
| Number of Bleeding, days | 4.96 ± 1.208 | | 4.89 ± 1.495 | | 4.92 ± 1.359 | |
| Number of Spotting, days | 1.80 ± 1.362 | | 1.87 ± 1.465 | | 1.84 ± 1.412 | |
| Smoking Status, n (%) | | | | | | |
| Never | 57 | (72.2) | 58 | (69.9) | 115 | (71.0) |
| Former | 16 | (20.3) | 19 | (22.9) | 35 | (21.6) |
| Current | 6 | (7.6) | 6 | (7.2) | 12 | (7.4) |
| Gravidity, n (%) | | | | | | |
| 0 | 47 | (59.5) | 45 | (54.2) | 92 | (56.8) |
| 1 | 12 | (15.2) | 15 | (18.1) | 27 | (16.7) |
| 2 | 13 | (16.5) | 15 | (18.1) | 28 | (17.3) |
| 3 | 4 | (5.1) | 5 | (6.0) | 9 | (5.6) |
| ≥4 | 3 | (3.8) | 3 | (3.6) | 6 | (3.7) |
| Parity, n (%) | | | | | | |
| 0 | 48 | (60.8) | 53 | (63.9) | 101 | (62.3) |
| 1 | 12 | (15.2) | 15 | (18.1) | 27 | (16.7) |
| 2 | 15 | (19.0) | 12 | (14.5) | 27 | (16.7) |
| 3 | 2 | (2.5) | 3 | (3.6) | 5 | (3.1) |
| ≥4 | 2 | (2.5) | 0 | (0.0) | 2 | (1.2) |
| Complications, n (%) | | | | | | |
| uterine fibroids/adenomyosis | 5 | (6.3) | 8 | (9.6) | 13 | (8.0) |
| uterine fibroids | 14 | (17.7) | 12 | (14.5) | 26 | (16.0) |
| adenomyosis | 22 | (27.8) | 22 | (26.5) | 44 | (27.2) |
| none | 38 | (48.1) | 41 | (49.4) | 79 | (48.8) |
| Clinical endometriosis, n (%) | 7 | (8.9) | 5 | (6.0) | 12 | (7.4) |

¹⁾mean ± standard deviation,
²⁾ range (min-max)

### Additional Inclusion and Exclusion Criteria

Japanese patients with regular menstrual cycles (25-38 days) for last 2 menstruation before randomization and BMI<30 kg/m² were included. Patients who have signed the informed consent form based on a full understanding of the trial were included.

Additional exclusion criteria were patients with undiagnosed abnormal vaginal bleeding within 6 months before the screening tests; patients aged ≥40 years with ovarian endometriomas for which the largest diameter was >10 cm; patients with ovarian endometriomas containing solid components; patients having undergone surgical treatment for endometriosis, adenomyosis and uterine fibroids by cyst puncture (such as transvaginal alcohol fixation), laparotomy or laparoscopy within 2 months before screening; patients who had had no improvement of their endometriosis symptoms (moderate or severe pelvic pain) with a combined oral contraceptive or hormone preparations containing progestin; patients who regularly use analgesics for medical reasons other than relief of endometriosis pain during the study (occasional use will be permitted; prophylaxis is not allowed); patients who had been diagnosed to undergo surgical treatment; patients with presence or history of hormone-related malignancy (non-melanoma skin cancer is allowed); patients with presence or history of deep vein thromboembolism, thrombophlebitis (except for surfaces), pulmonary thromboembolism, cerebrovascular disorder and coronary artery disease; patients who aged ≥35 years and smoked ≥15 cigarettes/day; patients with presence or history of migraine with aura; patients with valvular cardiac disease with pulmonary hypertension or atrial fibrillation, with a history of subacute bacterial endocarditis; patients with diabetes with vascular lesions (e.g. diabetic nephropathy, diabetic retinopathy); patients with known thrombogenic mutations (e.g. factor V Leiden; prothrombin mutation; protein S, protein C, and antithrombin deficiencies); patients with presence of anti-phospholipid antibody syndrome (e.g. Systemic lupus erythematosus with antiphospholipid antibody positive or unknown); patients who scheduled to undergo surgery within 4 weeks after informed consent signed, underwent surgery within 2 weeks before informed consent signed, or in a long-term rest state at the time of informed consent signed; patients with presence of severe hepatic impairment (e.g. acute viral hepatitis, severe cirrhosis); patients with hepatophyma, patients with presence of severe or acute kidney injury; patients who had a history of cardiac disease (e.g. uncomplicated valvular cardiac disease) or complications; patients with hypertension (except mild hypertension), severe dyslipidemia (e.g. severe familial abnormal p lipoproteinemia), otosclerosis, uncontrolled thyropathy; patients who clinically diagnosed with severe depression in the past year before screening or now; patients who had a history of jaundice, persistent pruritus, or herpes during pregnancy; patients being pregnant or possibly pregnant; patients who gave birth or had mid-pregnancy abortion within 6 weeks before screening; lactating women; patients who had predisposition to hypersensitivity to the components of the investigational product; patients who had a history of side effects or hypersensitivity that requires discontinuation of administration during sex steroid hormone therapy; patients being given drugs and derivatives that are thought to affect sex hormone secretion; patients who had taken hormone preparations containing progestin, estrogen, low-dose oral contraceptives, the combination drug of progesterone and estrogen, testosterone derivatives, estrogen antagonists, aromatase inhibitors, herbal medicine for dysmenorrhea, endometritis menstrual pain, anxiolytics or antispasmodics within a month before screening or had taken GnRH analogs within 2 months before screening; patients with current use or use within 1 month prior to subject randomization of drugs potentially triggering interactions with combined oral contraceptives. This includes, but is not limited to: CYP3A4 inducers, CYP3A4 inhibitors, HIV and/or HCV protease inhibitors, non-nucleoside reverse transcriptase inhibitors; patients who participated in other clinical trials within a month before the screening or administered other investigational products within 3 months before the screening; patients who desire to become pregnant or patients who do not agree to abstain from sexual intercourse or use contraceptive measures (use of barrier-type contraceptives (condoms for men or contraceptive pessaries) approved or certified in Japan) during this study; patients who deemed ineligible by the investigator or sub-investigator for other reasons.

### Treatment

Eligible subjects were randomly allocated to either the E4/DRSP or placebo group at an equivalent ratio balanced with baseline VAS (<60 mm or ≥60 mm) and complications (uterine fibroids and adenomyosis). Immediately after randomization, the subjects took one tablet daily from the first day of menstruation. In the E4/DRSP group, the subjects were treated with E4/DRSP in the cyclic regimen for six consecutive cycles, 24 weeks. The placebo group received oral placebo tablets daily for 28 days per cycle over six cycles. Stratification randomization codes were developed by the Interactive Web Response System using a permuted-block design, managed by an office independent of clinical investigators and other study stakeholders to ensure study blindness.

### Evaluating Endpoints

The primary endpoint was the VAS score change from baseline in the most severe EAPP following the six treatment cycles (Bourdel N, Alves J, Pickering G, Ramilo I, Roman H, Canis M. Systematic Review of Endometriosis Pain Assessment: How to Choose a Scale? Hum Reprod Update. 2015; 21:136; Gerlinger C, Schumacher U, Faustmann T, Colligs A, Schmitz F, Seitz C. 2010. Defining a Minimal Clinically Important Difference for Endometriosis-Associated Pelvic Pain Measured on a Visual Analog Scale: Analyses of Two Placebo-Controlled, Randomized Trials. Health Qual Life Outcomes. 2010; 8:138). During baseline observation and treatment periods, subjects were required to grade their most severe EAPP (lower abdominal pain/back pain) with VAS-installed electronic diary devices. The baseline was the most severe EAPP during the observation period before randomization. The secondary endpoints included: 1) Numeric Rating Scale (NRS) for pelvic, chronic, acute and defecation pain and dyspareunia, 2) responder rates accomplishing ≥30% or ≥50% reduction of the most severe EAPP VAS or averaged NRS from baseline to the fifth and sixth cycle, 3) gynecological examinations: cul-de-sac induration, pelvic tenderness and limitation of uterine mobility, 4) number and size of the ovarian endometriomas (two dimensional measurement) and endometrial thickness measured with TVUS, 5) interference with daily activity and sleep with a five-point scale, 6) seven-point scale rated by investigators (Clinical Global Impression of Improvement: CGI-I) and subjects (Patient Global Impression of Satisifaction: PGI-I), and 7) serum E2, P4, FSH, LH, CA125. All secondary variables were collected using patients' electronic diaries, and patients were questioned or examined by investigators at clinical sites.

Treatment-emergent adverse events (TEAEs) were monitored as safety endpoints throughout the study, and the investigators judged their severity and causality. Bleeding events were recorded by subjects using electronic diary devices.

### Statistical Analyses

The primary analysis was performed using Mixed effect Models for Repeated Measures to assess the point estimate of the group difference (E4/DRSP—placebo) with a two-sided 95% confidence interval (CI) of changes in VAS for the most severe EAPP from baseline to the sixth treatment cycle. Secondary endpoints were evaluated using Wilcoxon test, Fisher's exact test, and two-sided 95% CI. Any efficacy analysis was conducted on the full analysis set (FAS): subjects who received one or more study drugs and obtained VAS scores for EAPP. The safety analysis included the frequencies of any TEAEs, drug-related TEAEs, and severity and causality. This was performed using the safety analysis set of subjects taking at least one study tablet. All descriptive statistics were expressed as means±standard deviations. Statistical analyses were performed using SAS version 9.4 (SAS Institute Inc., NC, USA).

### Results

### Subjects

This study included 162 subjects from 25 clinical sites in Japan.

The FAS comprised 79 and 83 subjects in the E4/DRSP and the placebo groups, respectively. The demographic characteristics were comparable between the groups (Table 4). The average age and body mass index were 34.7 years and 21.4 kg/m², respectively. Of the 162 subjects, 44 (27.2%) and 26 (16.0%) presented with adenomyosis and uterine fibroids, respectively, and both complications were observed in 13 subjects (8.0%).

### Efficacy

On average, E4/DRSP reduced the most severe EAPP VAS score by −33.2 mm from the baseline following six treatment cycles. An average −22.2 mm decrease in pain intensity was observed in the placebo group. As presented in Table 5, the point estimate of the group difference was −8.5 mm (two-sided 95% CI: −16.1 to −0.9 mm) with significance following 24-week treatment (p=0.028).

TABLE 5
Change in VAS for the most severe EAPP from
baseline following 24 week treatment.

| | E4/DRSP | Placebo |
|---|---|---|
| | (n = 79) | (n = 83) |
| Baseline, VAS (mm) | 80.0 ± 14.93²⁾ | 76.4 ± 14.70 |
| 6th cycle (24 weeks), VAS (mm) | 47.2 ± 27.25 | 52.1 ± 24.40 |

Change from baseline to the 6th cycle, VAS (mm)

| | | |
|---|---|---|
| Observed | −33.2 ± 26.13 | −22.2 ± 23.44 |
| | (−39.2, −27.2)³⁾ | (−27.5, −16.9) |
| LSMean¹⁾ (MMRM) | −31.1 | −22.6 |
| | (−36.8, −25.4) | (−28.2, −17.0) |
| Group difference, LSMean | −8.5 | p = 0.028 |
| | (−16.1, −0.9) | |

¹⁾least square mean,
²⁾mean standard ± deviation,
³⁾two-sided 95% confidence interval

The responder rates of patients achieving ≥30% and ≥50% reduction of the most severe EAPP VAS from baseline during the fifth or sixth cycle were 53.2% and 36.4% in the E4/DRSP group, respectively, with significance to the placebo group.

The NRS grades indicated that E4/DRSP significantly relieved pain intensity but not dyspareunia and defecation. In the E4/DRSP group, the average NRS grades' responder rates were 55.8% and 36.4% for ≥30% and ≥50% reduction from the baseline grades, respectively, with significance to the placebo group.

After six treatment cycles, gynecological examinations showed objective improvement in the E4/DRSP group (Table 6). No worsened findings were diagnosed for cul-de-sac induration, and 23.1% of the subjects demonstrated significant improvements in the E4/DRSP group. A similar result was obtained for the limitation of uterine mobility. E4/DRSP also prevented the progression of pelvic tenderness compared to the proportion of subjects who worsened in the placebo group (E4/DRSP 1.3% vs. placebo 12.0%) (FIG. 5A). Substantial improvements were also observed in the subjects with adenomyosis (FIG. 5B). The volume of the largest ovarian endometrioma was reduced by approximately 45.0% in the E4/DRSP group compared to that in the placebo group. Ovarian endometriomas also disappeared in 7.7% of E4/DRSP group subjects. Serum CA125 levels recovered to the normal range (<35 U/mL) in 19.2% of the E4/DRSP group and only 2.4% in the placebo group.

TABLE 6
Frequency of Douglas Fossa Induration, Limitation of Uterine Mobility
and Pelvic Tenderness stratified by Adenomyosis (FAS).

| | | | | FSN-013 | Placebo |
|---|---|---|---|---|---|
| Adenomyosis | Parameter | Visit | Category | n (%) | n (%) |
| No | Douglas Fossa | Visit 10/ | Improved | 10 (19.6) | 4 (7.5) |
| | Induration | Discontinuation | Stable | 41 (80.4) | 45 (84.9) |
| | | | Worsened | 0 (0.0) | 4 (7.5) |
| | Limitation of | Visit 10/ | Improved | 9 (17.6) | 3 (5.7) |
| | Uterine | Discontinuation | Stable | 42 (82.4) | 48 (90.6) |
| | Mobility | | Worsened | 0 (0.0) | 2 (3.8) |
| | Pelvic | Visit 10/ | Improved | 8 (15.7) | 8 (15.1) |
| | Tenderness | Discontinuation | Stable | 43 (84.3) | 38 (71.7) |
| | | | Worsened | 0 (0.0) | 7 (13.2) |
| Yes | Douglas Fossa | Visit 10/ | Improved | 8 (29.6) | 2 (6.7) |
| | Induration | Discontinuation | Stable | 19 (70.4) | 27 (90.0) |
| | | | Worsened | 0 (0.0) | 1 (3.3) |
| | Limitation of | Visit 10/ | Improved | 8 (29.6) | 2 (6.7) |
| | Uterine | Discontinuation | Stable | 18 (66.7) | 27 (90.0) |
| | Mobility | | Worsened | 1 (3.7) | 1 (3.3) |
| | Pelvic | Visit 10/ | Improved | 9 (33.3) | 7 (23.3) |
| | Tenderness | Discontinuation | Stable | 17 (63.0) | 20 (66.7) |
| | | | Worsened | 1 (3.7) | 3 (10.0) |

* Based on 4-level rating scale results (None, Mild, Moderate, Severe), categorized as “Improved” or “Worsened” for changes of 1 level or more before and after administration, and “Stable” for changes of less than 1 level before and after administration. Baseline: Visit 4.

In the E4/DRSP group, daily activities did not improve significantly. Nonetheless, six cycles of E4/DRSP treatment resulted in no subjects rating “extremely disturbed” and reduced the proportion of “quite disturbed” by 9.0%, different from the placebo group. Sleep disturbance was significantly improved in the E4/DRSP group, and the proportion of subjects rating “not disturbed at all” increased by 52.6%. Considerable improvements were observed in global impressions, the proportion of which was approximately 45% in the E4/DRSP group. Serum endocrine hormone levels decreased in the E4/DRSP group throughout the study. Endometrial thicknesses changed from 9.81±3.66 mm at baseline to 4.83±2.48 mm at 24 weeks in the E4/DRSP group.

### Safety

TEAEs were reported in 77 of 79 subjects (97.5%) in the E4/DRSP group and in 72 of 83 subjects (86.7%) in the placebo group. In the E4/DRSP group, intermenstrual bleeding events commonly occurred and declined with treatment cycles: 51.9% during the first cycle and 24.7% after treatment. Spotting was the predominant event after the third treatment cycle, accounting for approximately 50% of the bleeding events. Intermenstrual bleeding/spotting occurred less than 1 day after the second treatment cycle. The study drug-related TEAEs were similar to those commonly reported for OCPs, including nausea (6.3%), abdominal pain (2.5%), diarrhea (2.5%), somnolence (6.3%) and headache (6.3%) in the E4/DRSP group. One subject in the placebo group discontinued treatment because of an increased D-dimer level. In particular, fewer differential changes were observed in the proportion of subjects with hemostasis parameters outside the reference ranges between the E4/DRSP and placebo groups. No deaths or other serious AEs occurred during the treatment. No clinically relevant changes were observed in the other safety endpoints.

### DISCUSSION

In this study, E4/DRSP improved the most severe EAPP in the cyclic regimen, similar to EE/DRSP in a flexible extended regimen (Harada T, Kosaka S, Elliesen J, Yasuda M, Ito M, Momoeda M. Ethinylestradiol 20 g/drospirenone 3 mg in a flexible extended regimen for the management of endometriosis-associated pelvic pain: a randomized controlled trial. Fertil Steril 2017; 108:798-805), and its superiority to placebo was confirmed. Responder rates also demonstrated more robust findings on EAPP alleviation (Dworkin R H, Turk D C, Farrar J T, Haythornthwaite J A, Jensen M P, Katz N P, et al. Core outcome measures for chronic pain clinical trials: IMMPACT recommendations. Pain 2005; 113:9-19), which suggested that approximately 40% of subjects resulted in ≥50% reduction of pain intensity from baseline. Moreover, pain intensity was reduced by <40 mm, a target of chronic pain management (Carol S. Burckhardt and Kim D. Jones. Adult measures of pain the McGill Pain Questionnaire (MPQ), Rheumatoid Arthritis Pain Scale (RAPS), Short-Form McGill Pain Questionnaire (SF-MPQ), Verbal Descriptive Scale (VDS), Visual Analog Scale (VAS), and West Haven-Yale Multidisciplinary Pain Inventory (WHYMPI). Arthritis Care Res 2003; 49:S96-S104), in ≥50% of subjects treated with E4/DRSP for 24 weeks.

Objective findings of gynecological examinations revealed significant improvements in cul-de-sac induration, pelvic tenderness, and limitation of uterine mobility in the E4/DRSP group. The volume of ovarian endometrioma was significantly decreased in the E4/DRSP group, similar to the EE/DRSP and EE/norethisterone groups (Taniguchi F, Enatsu A, Ota I, Toda T, Arata K, Harada T. Effects of low dose oral contraceptive pill containing drospirenone/ethinylestradiol in patients with endometrioma. Eur J Obstet Gynecol Reprod Biol 2015; 191:116-20.; Harada T, Momoeda M, Taketani Y, Hoshiai H, Terakawa N. Low-dose oral contraceptive pill for dysmenorrhea associated with endometriosis: a placebo-controlled, double-blind, randomized trial. Fertil Steril 2008; 90:1583-8). These treatment benefits are best exemplified by the considerable advantages of QoL-related questionnaires and global impression ratings in the E4/DRSP group.

Safety evaluations revealed that intermenstrual bleeding events were the most frequently reported TEAE associated with E4/DRSP. The frequency decreased with the number of cycles, consistent with previous Phase III studies involving 2,234 participants from outside Japan (Kaunitz A M, Achilles S L, Zatik J, Weyers S, Piltonen T, Suturina L, et al. Pooled analysis of two phase 3 trials evaluating the effects of a novel combined oral contraceptive containing estetrol/drospirenone on bleeding patterns in healthy women. Contraception 2022; 116:29-36). Other reported TEAEs are well-known for OCPs, such as nausea and headache. No VTE was reported, and the proportion of subjects with D-dimer over the upper range was comparable to that of the placebo group, suggesting less effect of E4/DRSP on hemostasis parameters.

E4/DRSP fulfilled the following requirements for treating endometriosis: alleviation of EAPP, improvement in objective gynecological findings, recovery of QoL and global impressions. No safety concerns were raised, including hemostasis and bleeding patterns.

### CONCLUSIONS

This study demonstrated that E4/DRSP is clinically effective for EAPP treatment, with improved objective gynecological findings, QoL and global impressions in patients with endometriosis. Therefore, E4/DRSP should be considered a first-line endometriosis treatment.

1-18. (canceled)

19. A method of relieving pain associated with endometriosis, comprising administering to a subject in need thereof a pharmaceutical composition comprising from about 13.5 mg to about 16.5 mg estetrol or a hydrate thereof and from about 2.5 mg to about 3.5 mg drospirenone.

20. The method of claim 19, wherein the pain is pain during a non-withdrawal bleeding period.

21. The method of claim 19, wherein prior to treatment the subject has a Visual Analogue Scale (VAS) score of greater than about 40 mm, greater than about 50 mm, greater than about 60 mm, or greater than about 70 mm.

22. The method of claim 19, wherein the pain is resulting from endometrial tissue engrafts outside the uterine cavity in the pelvis.

23. The method of claim 19, wherein the method is effective to reduce pain as reflected in an improvement in the subject's Clinician Global Impressions-Improvement Scale (CGI-I Scale).

24. The method of claim 19, wherein the method is effective to reduce pain as reflected in an improvement graded “significantly satisfied or more” in the subject's Patient Global Impressions-Improvement Scale (PGI-I Scale).

25. The method of claim 19, wherein the method exhibits a responder proportion of at least about 60%.

26. The method of claim 19, wherein the subject has been diagnosed as having endometriosis by laparotomy/laparoscopy, and/or as having ovarian chocolate cysts as assessed by Transvaginal Ultrasound (TVUS) and/or Magnetic Resonance Imaging (MRI).

27. The method of claim 19, wherein the method comprises daily administration of the composition in cycles comprising 21-28 administration days, optionally in cycles comprising 24 administration days.

28. The method of claim 27, wherein each cycle further comprises an administration-free interval of 4-7 days, optionally wherein the administration-free interval is 4 days.

29. The method of claim 27, wherein the method is effective to reduce paid as reflected in a decrease in the subject's VAS score after a second or third cycle, as compared to the subject's VAS score prior to treatment, optionally wherein said decrease in VAS score is maintained substantially stably after the second or third cycle, respectively.

30. The method of claim 19, wherein the method results in one or more selected from improvement in severity of Douglas induration, improvement in restriction of uterine mobility, improvement in pelvic tenderness, reduced size of ovarian chocolate cysts as assessed by TVUS or MRI, and/or reduced number of ovarian chocolate cysts as assessed by TVUS or MRI.

31. The method of claim 19, wherein the method is associated with fewer Treatment Emergent Adverse Events (TEAE) as compared to administration of a fixed-dose combination tablet comprising 20 μg ethinylestradiol and 3 mg drospirenone, wherein the TEAE are selected from intermenstrual bleeding, headache, nausea, and heavy menstrual bleeding.

32. The method of claim 19, wherein the subject has one or both of adenomyosis and uterine fibroids, in addition to endometriosis.

33. A method of treating a subject suffering from pelvic pain, comprising administering to the subject a pharmaceutical composition comprising from about 13.5 mg to about 16.5 mg estetrol or a hydrate thereof and from about 2.5 mg to about 3.5 mg drospirenone.

34. The method of claim 33, wherein the subject has pelvic pain defined by a Visual Analogue Scale (VAS) score of greater than about 40 mm, greater than about 50 mm, greater than about 60 mm, or greater than about 70 mm prior to treatment.

35. The method according to claim 33, wherein said pain is selected from chronic pelvic pain, lower abdominal pain, low back pain, defecation pain, sexual intercourse pain, and pain caused by adhesion of endometrium in the Douglas pouch.

36. A method of relieving pain associated with endometriosis in a subject in need thereof, comprising:
identifying a subject who has pelvic pain prior to treatment with a VAS score of greater than about 40 mm, about 50 mm, about 60 mm, or about 70 mm, and
administering to the identified subject a pharmaceutical composition comprising from about 13.5 mg to about 16.5 mg estetrol or estetrol monohydrate and about 2.5 mg to about 3.5 mg drospirenone.
